(19) United States
(12) Patent Application Publication
(10) Pub. No.: US 2026/0284408 A1
(43) Pub. Date: Sep. 24, 2026

(54) ELECTRODE ASSEMBLY

(71) Applicant: Berlin Heals GmbH, Berlin (DE)

(72) Inventor: Thomas Töllner, Ketzin (DE)

(21) Appl. No.: 19/573,714

(22) Filed: Mar. 20, 2026

(30) Foreign Application Priority Data
Mar. 21, 2025 (EP) 25165471.1

(51) Int. Cl. A61N 1/362; A61N 1/04; A61N 1/05; A61N 1/375

(52) U.S. Cl. CPC A61N 1/3628; A61N 1/0492; A61N 1/0573; A61N 1/3627; A61N 1/3756; A61N 1/3758

(57) ABSTRACT

The present invention relates to an electrode assembly having multiple electrodes, the multiple electrodes comprising a first electrode and at least one additional electrode, wherein each of the electrodes comprises an electrically active part, and a control unit being supplied with power by a power supply unit, wherein the control unit is electrically connected to each of the electrodes, wherein the control unit is configured to establish a potential difference between an anode and a cathode, wherein the anode is represented by one or more of the electrically active parts of the multiple electrodes and the cathode is represented by one or more of the remaining electrically active parts of the multiple electrodes, such that an electric current with a finite DC component is induced between the anode and cathode, the control unit being configured to set the electric current to a preset value by regulating the potential difference between the anode and the cathode, characterized in that the control unit and at least one of the multiple electrodes are positioned with a finite distance to an internal organ, such as a heart, wherein the relative orientation of the anode and the cathode is implemented such that at least a partial volume of the heart is situated inside an induced electric current path between the anode and the cathode.

### CROSS-REFERENCE TO RELATED APPLICATIONS

This application claims priority to European Application No. 25165471.1, filed on Mar. 21, 2025, the entire contents of which are hereby incorporated by reference.

### TECHNICAL FIELD

The present invention relates to an electrode assembly. Further, the present invention relates to an implantable electrode assembly. More particularly, it relates to an implantable and/or extracorporeal electrode assembly for treating heart failure.

### TECHNOLOGICAL BACKGROUND

It has been shown that the application of microcurrent directly to the heart leads to an improvement of cardiac function in patients with heart failure (Kosevic, Dragana et al. “Cardio-microcurrent device for chronic heart failure: first-in-human clinical study.” ESC heart failure vol. 8,2(2021 ): 962-970). For this purpose, the microcurrent was applied between an epicardial patch lead placed extrapericardially or intrapericardially over the free wall of the left ventricle and a coil lead placed in the right ventricle. Examples of such patch leads are disclosed for example in WO 2016/016438 or in WO 2006/10132.

Furthermore, it could be shown that on the level of cultured cardiomyocytes, the application of microcurrent modulates myofibroblasts for cardiac repair and regeneration (Somesh DB et al. “Microcurrent-Mediated Modulation of Myofibroblasts for Cardiac Repair and Regeneration”. International Journal of Molecular Sciences. 2024, 25, 3268. doi. org/10.3390/ijms25063268).

For the placement of the patch lead on the heart, the thorax is surgically opened by an invasive procedure that carries a high degree of risk which is furthermore less readily accepted by patients. It also requires collaboration between cardiologists and cardiothoracic surgeons, which makes the procedure more laborious and difficult to plan and carry out. Thus, there is a need for an electrode assembly system that can be transplanted or applied more easily, with less risks and that is capable of effective treatment of the heart. Similar considerations apply to treatments of other internal organs.

### SUMMARY OF THE INVENTION

In order for the present description to be more readily understood, certain terms are first defined. Additional definitions are set forth throughout this summary and the detailed description.

It is to be noted that the term “a” or “an” entity refers to one or more of that entity; for example, “a cathode,” is understood to represent one or more cathodes. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, Korpas, David. Implantable cardiac devices technology. Berlin: Springer, 2013; Troutman, Leslie. “Dictionary of Medical Technology.” RQ 32.3(1993 ): 421-423, provide a general dictionary of many of the terms used in this disclosure as an exemplary representation of the common general knowledge of a person skilled in the art.

Units, prefixes, and symbols are denoted in their Système International d'Unités (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety. The term “about” is used herein to mean approximately, roughly, around, or in the regions of. When the term “about” is used in conjunction with a numerical range, it modifies that range by extending the boundaries above and below the numerical values set forth, wherein the value or range is commonly interpreted as being as accurate as the method used to measure it. In general, the term “about” can modify a numerical value above and below the stated value by a variance of, e.g., 10 percent, up or down (higher or lower).

In particular, using such terms as “about” implies that a certain effect or result can be obtained within a certain tolerance and the skilled person knows how to obtain that tolerance.

The terms “biocompatibility” or “biocompatible” describe the appropriate biological requirements of a biomaterial or biomaterials used in a medical device as well as the ability of a material to perform with an appropriate host response in a specific application. In the context of the invention, the term “biocompatibility” specifically means the ability of the material of the assembly to function in vivo without eliciting detrimental local or systemic responses in the body. The term “biostability” or “biostable” refers to the ability of a material to maintain its physical and chemical integrity after implantation into a living tissue.

In order to address the need as explained above, the object of the present invention is addressed by providing an electrode assembly that is less invasive, enables effective treatment of the heart or another internal organ and potentially makes the system fully percutaneously implantable or at least partly extracorporeally applicable.

The invention provides an electrode assembly having multiple electrodes, the multiple electrodes comprising a first electrode and at least one additional electrode, wherein each of the electrodes comprises an electrically active part, and a control unit being supplied with power by a power supply unit, wherein the control unit is electrically connected to each of the electrodes, wherein the control unit is configured to establish a potential difference between an anode and a cathode, wherein the anode is represented by one or more of the electrically active parts of the multiple electrodes and the cathode is represented by one or more of the remaining electrically active parts of the multiple electrodes, such that an electric current with a finite direct current (DC) component is induced between the anode and the cathode, the control unit being configured to set the electric current to a preset value by regulating the potential difference between the anode and the cathode, characterized in that the control unit and at least one of the multiple electrodes are positioned with a finite distance to an internal organ, wherein the relative orientation of the anode and the cathode is implemented such that at least a partial volume of the internal organ is situated inside an induced electric current path between the anode and the cathode.

According to a first embodiment of the invention, an implantable electrode assembly is provided having multiple electrodes, the multiple electrodes comprising an implantable first electrode and at least one implantable additional electrode, wherein each of the electrodes comprises an electrically active part, and a control unit being supplied with power by a power supply unit, wherein the control unit is electrically connected to each of the electrodes, wherein the control unit is configured to establish a potential difference between an anode and a cathode, wherein the anode is represented by one or more of the electrically active parts of the multiple electrodes and the cathode is represented by one or more of the remaining electrically active parts of the multiple electrodes, such that an electric current with a finite direct current (DC) component is induced between the anode and the cathode, the control unit being configured to set the electric current to a preset value by regulating the potential difference between the anode and the cathode, characterized in that the control unit and at least one of the multiple electrodes are positioned with a finite distance to a heart, wherein the relative orientation of the anode and the cathode is implemented such that at least a partial volume of the heart is situated inside an induced electric current path between the anode and the cathode.

According to a second embodiment of the invention, an electrode assembly is provided having multiple electrodes, the multiple electrodes comprising at least one extracorporeal electrode and at least one additional electrode, wherein each of the electrodes comprises an electrically active part, and a control unit being supplied with power by a power supply unit, wherein the control unit is electrically connected to each of the electrodes, wherein the control unit is configured to establish a potential difference between an anode and a cathode, wherein the anode is represented by one or more of the electrically active parts of the multiple electrodes and the cathode is represented by one or more of the remaining electrically active parts of the multiple electrodes, such that an electric current with a finite direct current (DC) component is induced between the anode and the cathode, the control unit being configured to set the electric current to a preset value by regulating the potential difference between the anode and the cathode, characterized in that the control unit and the at least one extracorporeal electrode is positioned with a finite distance to an internal organ, wherein the relative orientation of the anode and the cathode is implemented such that at least a partial volume of the internal organ is situated inside an induced electric current path between the anode and the cathode.

The above first embodiment of the invention is characterized in that all of the electrodes are specified as being implantable and that the internal organ is a heart. The above second embodiment of the invention is further specified in comparison to the more general invention described herein in that at least one electrode is specified as being extracorporeal, which is also the one with the finite distance to the internal organ. The following description of embodiments, aspects and implementations apply to the generally described invention as well as to either or both of the above first and second embodiments of the invention, if applicable.

In a preferred embodiment according to the invention, the internal organ may be a human heart. The invention is however, also applicable to any other internal organ of the human body.

The electrode assembly of the present invention generally comprises a control unit and at least two electrodes. The components of the assembly may individually be implantable, extracorporeal or a combination thereof.

In a preferred embodiment of the invention, the electrode assembly is fully implantable, i.e. every component including the control unit, the first electrode and the at least one additional electrode(s) are implantable.

In an embodiment featuring a combination of implantable and extracorporeal components, the electrode assembly according to the invention may for example comprise one implantable electrode and at least one extracorporeal electrode. Additionally, the assembly may comprise an implantable and/or extracorporeal control unit.

Accordingly, the electrodes of the present invention may be implantable or extracorporeal. An electrode may be considered implantable if it is positioned within the human body. An electrode may be considered extracorporeal if it is positioned externally to the human body or placed on the outside of and in contact with the skin. The implantable or extracorporeal electrodes may generally comprise an electrical conductor and optionally an electrical insulation being arranged around portions of the electrical conductors of the electrodes.

In the case of implantable electrodes, the electrical conductor and/or the electrical insulation may be any kind of material that are sufficiently biocompatible within the human body. They may further be considered as being made of non-corrosive and/or radiopaque materials. In particular, the electrically conducting material of the electrodes may comprise a material with high corrosive resistance, preferably a metal or metal compound with high corrosive resistance, for example a compound of Platinum-Iridium (PtIr). The parts of at least one electrode of the electrode assembly intended for carrying electric current, i.e. the electrically conducting components within the electrically conducive path or in other words the electric circuit parts of the respective electrode of the assembly, may be comprised of the same electrically conducting (metallic) material with high corrosive resistance like PtIr. Preferably, all of the electrically conducting components within the electrically conducive path of each of the electrodes used in the assembly may comprise the same electrically conducting (metallic) material like PtIr.

The implantable electrodes may not be restricted in size or shape as long as they are suitable to be implanted in the human body, particularly implantable in close proximity to or inside the human heart. An electrode may be implantable if there are no or only minor damages to the human body or if there is no or only minor adverse interaction therewith. An electrode may be implantable when the implantation of the electrode does not or only do minor damage to the human body when being implanted for a time span of at least longer than one hour, more preferably longer than one day, one week, one month or one year.

Extracorporeal electrodes may be in direct contact with the outer skin of the human body. Such electrodes may thus not be restricted by the same biocompatibility requirements as the implantable electrodes. Instead, the extracorporeal electrodes may only be sufficiently biocompatible to not be harmful to the outer skin of the human body to which they are applied. Therefore, extracorporeal electrodes may be easier to manufacture.

Extracorporeal electrodes may comprise adhesive electrodes, dry microneedle electrodes (MNEs), such as a PEDOT: PSS coating and/or breathable electrodes. The electrical transcutaneous path may be configured to be as conductive as possible. Extracorporeal electrodes with low (contact) resistance may thus be used.

The at least one additional electrode may be only one additional electrode, in which case it may be referred to as a second electrode. In case there is more than one additional electrode, the further electrodes may be referred to as a third electrode, a fourth electrode, a fifth electrode and so forth.

The potential difference may be referred to as a voltage. Dependent on whether the sign of the applied voltage between certain (groups of) electrodes is positive or negative, one or more of the electrodes will be charged negatively, while the remaining one or more electrodes will be charged positively. The negatively charged electrically active part(s) of the electrode(s) may be referred to as a cathode, while the positively charged electrically active part(s) the remaining electrode(s) will be referred to as an anode. The sign and strength of the applied voltage may be regulated by the control unit.

The electrically active part of each of the multiple electrodes may generally refer to portions of the electrodes from which the electric current that is induced by the potential difference is mainly emitting from or entering into. The electrically active parts may thus be uninsulated or less insulated portions of the respective electrodes, from which an electric current can flow to the respective other electrode. Additionally, the electrodes may be geometrically structured such that the electric current is maximal/large at the electrically active part of the electrode and falls below a threshold current elsewhere on the electrode.

Correspondingly, the remaining portion of the electrode (“elsewhere”) may be referred to as an electrically inactive part. This part may be configured to be no or a negligible source of electric current. Electrically inactive parts may be the insulated portions of an electrode that are not configured to be a main source of electric current flow.

Unless specified otherwise, the term “electrode” as used herein may be referred to as the electrically active part of an electrode.

Unless specified otherwise, the term “current” refers to electric current and may be direct current (DC) or alternating current (AC). The electric current may also be understood as an electric current density. During the intended medical treatment, the current density between electrodes being cathode and anode will be adjusted to preferably 0.1 to 100 μA/cm², more preferably to 0.5 to 10 μA/cm².

Although the abbreviation DC may generally refer to “direct current”, in the context of the present disclosure, DC may generally refer to constant or quasi-stationary components of an electric current. Accordingly, the electric current with a finite DC component may be understood as an electric current applied via the control unit having a finite component of direct electric current that is constant or at least quasi-stationary in time. In a more specific embodiment according to the invention, a finite DC component may be understood as any frequency component of an electric current that is below 1 Hz. In other embodiments, it may be referred to frequencies below 0.1 Hz or even below 0.001 Hz or 0.0001 Hz. Correspondingly, frequency components of electric currents that lie above one of either of these values may be referred to as AC herein.

The term “finite” as used in “finite DC component” herein may be understood as “non-zero”. Correspondingly, a finite DC component is characterized in that the electric current induced between the cathode and anode has a non-zero component characterized as being DC as defined herein.

The control unit may have an integrated radio transceiver built inside or alternatively a separate radio transceiver electrically connected to the control unit. The radio transceiver may have the capability of wirelessly communicating with an external device such as a portable user terminal for remote programming of the control unit. Further, the radio transceiver may be capable of transmitting data to an external device. The data may comprise measurements of applied voltage, current, current density, resistance, impedance, pressure, temperature and the like.

The control unit may be a programmable voltage source and/or a current source. In either case, a voltage may be controlled by the control unit. In the case of the control unit being a voltage source, the control unit may be configured to regulate the voltage. In the case of the control unit being a current source, the control unit may be configured to regulate the voltage such that a predetermined electric current can be applied.

The preset value of the DC component of the electric current may be a preset value of an amperage or a current density flowing between the electrodes.

The control unit may be programmable to predetermine a time interval within which the potential difference is maintained to obtain direct electric current (DC) flow, which can range from some minutes, over an interval of 30 minutes or an hour until a number of hours, days or months. After a predefined time, the current direction can be inverted, and a similar time interval may be provided after such a first time interval. This changes the direction of the flow of the current. This sequence of change of current flow inversion can be continued for prolonged periods of time, e.g. for up to several months or even years.

It may also be possible to change the current strength while inverting the current flow since the impedance between electrodes may be dependent on the direction of the current flow. The amount of the direct current flow may be predetermined to be far below the stimulation threshold, especially chosen to have a current density of 0.1 μA/cm² to 1 mA/cm². The control unit can comprise a control to maintain the current density below a maximum threshold.

Inverting a current flow may be executed quasi-stationary, i.e., decreasing the current density over several minutes to zero and raising it with the opposite leading sign to the predetermined new direct current density level to avoid any rhythm disturbances which can potentially lead to dys- or arrhythmia.

The control unit may be programmable to apply arbitrary voltage waveforms and/or arbitrary current waveforms. The waveforms may include sine waves, saw waves, square waves, triangle waves and pulses of different forms. In a particularly preferred embodiment, the control unit may be configured to apply combinations of DC voltage/current and AC voltage/current.

The control unit may be configured to apply a DC component of voltage or current together with an AC component of voltage or current. More particularly, the absolute voltage or current value of the DC component may be larger than the absolute peak amplitude of the respective voltage or current value of the AC component. This way, a DC voltage or current may be modulated by the AC component without inverting the polarity of the applied voltage or current.

It may be understood that the application of a DC component of electric current according to the invention is fundamentally different to the application of non-stationary, alternating currents or current pulses. In particular, current pulses are widely used for pacemaking or defibrillation purposes. Such current pulses have typical pulse widths of 0.5 ms to 1 ms (but are not limited thereto). It is thus clear for the person skilled in the art that such pulses used in the prior art contain a wide range of high frequency components, which stands in contrast to the characteristics of the presently used DC component of electric current. Of course, using AC components of electric current for pacemaking or defibrillation purposes might still be included herein.

Pacemakers have the function of emitting electrical impulses to the heart which trigger contractions of the heart if the heart's own impulse generators (e.g. sinus node) are not functional for whatever reason. To ensure that these impulses are delivered at the right time via the implanted electrode, a pacemaker is configured to constantly measure the heart's electrocardiogram (ECG) and check whether or not the heart is generating its own impulses that can trigger a contraction. At the moment when the heart's own impulses are not registered, the pacemaker emits an electrical impulse of sufficient strength to trigger a contraction.

Pacemaker electrodes therefore have two functions: (i)—sensing the cardiac (intrinsic) ECG, and (ii)—conditionally emitting an electrical impulse that triggers a contraction.

Sensing the ECG is an important function in pacemaker therapy. To prevent oversensing (when inappropriate electrical activity is recognized as appropriate intrinsic cardiac activity) or undersensing (a lack of perception [permanent or intermittent] of cardiac signals), the electronic input circuit responsible for sensing has a filter characteristic that is typically tailored to the real ECG and is impermeable to direct current. Similarly, the pulses emitted by a pacemaker have no direct current component.

Defibrillators on the other hand are devices designed to detect arrhythmias (e.g. ventricular tachycardia or ventricular fibrillation) that are so pronounced that the heart can no longer pump sufficiently. If these are detected, defibrillators are configured to emit a strong electrical impulse (shock) to restore the heart's rhythm.

As with pacemakers, the ECG of the heart is continuously monitored via the sensor electrodes in order to detect corresponding ECG changes. Since it should be avoided as far as possible that shocks are erroneously delivered or that a necessary shock is not delivered, the electronic input circuits for sensing are equipped with appropriate filters that do not allow any direct current to pass. The shock pulses can be configured differently, but also have no direct current component.

Accordingly, current pulses according to the prior art may not be confused with the stationary or quasi-stationary direct currents discussed herein. In fact, the application of a direct current according to the invention has particular advantages for heart treatment as discussed in Dragana Kosevic et al.: “Cardio-microcurrent device for chronic heart failure: first-in-human clinical study”, ESC HEART FAILURE, vol. 8, no. 2, 9 Feb. 2021.

The present invention may thus specifically exclude devices that operate on a pure pacemaking or defibrillating basis. More particularly, any implantable device that does not use DC currents or at least finite DC components for treating a heart may be explicitly excluded from the subject-matter of the present invention.

An electrode being positioned with a finite distance to the heart or another internal organ may be referred to any position of an electrode that exhibits no direct contact to the heart or another internal organ. In specific embodiments, the finite distance to the heart or another internal organ may refer to a distance above 0.1 cm, 0.2 cm, 0.3 cm, 0.4 cm, 0.5 cm, 0.6 cm, 0.7 cm, 0.8 cm, 0.9 cm or above 1 cm. In more specific embodiments, the finite distance to the heart or another internal organ may mean a distance above 2 cm, 3 cm, 4 cm, 5 cm, 6 cm, 7 cm, 8 cm, 9 cm or 10 cm. In even more specific embodiments, the finite distance to the heart or another internal organ may mean a distance above 15 cm, 20 cm, 25 cm, 30 cm or 35 cm. Additionally or alternatively, the finite distance to the heart or another internal organ may refer to a distance range between 1 cm and 50 cm, preferably between 1 cm and 35 cm, more preferably between 5 cm and 35 cm, even more preferably between 10 cm and 35 cm.

The term “finite” as used in “finite distance to the heart” or “finite distance to the internal organ” as used herein may be understood as “non-zero distance to the internal organ/heart”. Correspondingly, a finite distance to the heart may be understood as a distance that is different from zero. An electrode having a finite distance to the internal organ or heart may thus exclude direct contact thereto.

When referring specifically to a “direct contact to the heart”, this may be understood by the skilled person as any immediate or direct contact of an electrode to the epicardium or the pericardium of the heart. Correspondingly, when referring to the “outside of the heart” or an “outer wall” of the heart, this may be understood as the outmost layer of the epicardium or the pericardium.

Further, a direct contact of an electrode to the heart or another internal organ may be given as soon as at least a part of the electrode exhibits direct contact according to the above definitions. Accordingly, a finite distance of an electrode to the heart may only be given if the electrode as a whole, or at least the active part of the electrode, exhibits a finite distance according to the definitions given above.

According to a preferred embodiment of the invention, the finite distance to the heart or another internal organ may refer to distances above 1 cm from the pericardium or the epicardium or another internal organ.

The relative orientation of the anode and the cathode is arranged such that at least a partial volume of the internal organ, such as a heart, is situated inside an induced electric current path between the anode and the cathode. Generally, the induced electric current path between a cathode and an anode, each of the cathode and the anode being one or more electrodes implanted in the human body, may be a rather complex spatial distribution and highly dependent on the relative orientation of the respective electrodes and the body parts lying therebetween. Thus, the induced current path may be characterized or defined by a spatial current density distribution emerging between the cathode and anode which will be readily apparent to a skilled person. Using this definition of the induced current path, the path may be spatially limited by only considering those parts of the distribution that are above a threshold value of current density. The threshold value for the current density may be 0.1 μA/cm² or preferably 0.5 μA/cm².

Thus, according to the invention, the partial volume of the internal organ, such as a heart, may be situated within the induced electric current path if the electric current density along the induced electric current path lies at least above the threshold value.

According to the invention, the control unit may either be an implantable control unit or an external/extracorporeal control unit. In case that the control unit is implanted, it may be arranged with finite distance to the internal organ, such as a heart. An implantable control unit may fulfill the same requirements for biocompatibility as the implantable electrodes. An extracorporeal control unit may be fully external to the human body and thus have no specific requirements with regard to biocompatibility.

According to the invention, two or more implantable and/or extracorporeal electrodes may be considered. One or more of the electrodes may be used as a cathode (or alternatively as an anode), while the remaining one or more electrodes may be used as an anode (or alternatively as a counter-cathode, if the other electrode acts as anode), respectively. In this case, the induced current path between the cathode and anode according to the invention may be understood as the sum of the current distributions between the cathode and the anode. In other words, the current distribution between the anode and cathode according to the invention may refer to the current distributions between each cathode-anode pairing. Hence, in a configuration comprising more than two electrodes, the sum of any possible cathode-anode combination may be considered to define the current distribution between the anode and cathode as defined according to the invention.

In a first implementation of the present aspect, only two electrodes are considered. For example, there may be a first electrode being an anode and a second electrode being a cathode (or vice versa). Then, the current path between the electrically active parts according to the invention may simply be the current distribution between the electrically active part of the first electrode and the electrically active part of the second electrode.

In a second implementation of the present aspect, three electrodes may be considered. For example, there may be a first electrode being an anode, and a second and third electrode being a cathode (or vice versa). At least one of the three electrodes is placed with finite distance to the internal organ, such as the heart, while the remaining electrodes may be individually placed with either zero distance or finite distance to the internal organ, such as the heart. The induced current path between the electrically active parts may be the sum of the current distributions between the electrically active part of the first electrode and the electrically active part of the second electrode plus that between the electrically active part of the first electrode and the electrically active part of the third electrode.

In a third implementation of the present aspect, a configuration comprising four electrodes may be considered, i.e. a first, second, third and fourth electrode. In one embodiment, the first and second electrode may be cathodes, while the third and fourth electrodes may be anodes (or vice versa). At least one of the three electrodes is placed with finite distance to the internal organ, such as the heart, while the remaining electrodes may be individually placed with either zero distance or finite distance to the internal organ, such as the heart. The induced current path between the anode and cathode may be given by the sum of current distributions between the first and third, the first and fourth as well as the second and third, and the second and fourth electrode.

The present invention aims at providing an implantable and/or extracorporeal electrode assembly that features at least one electrode that does not have to be applied directly on or inside an internal organ, such as the heart, but is rather positioned away from the internal organ/heart and does not have any direct contact thereto. In some embodiments, the at least one electrode with finite distance to the internal organ/heart is extracorporeal, more specifically it may be applied to the outside of the skin. This is generally advantageous as it lowers the invasiveness of the implanting procedure of such electrode assembly for a patient as compared to electrode assemblies having direct contact to the internal organ/heart for all or part of the electrodes used in the assembly. For the particular electrode being positioned with finite distance to an internal organ, such as the heart, according to the present invention, this may allow omitting the necessity to involve both a thorax surgeon and a cardiologist, wherein the thorax may not have to be surgically opened. Instead, only the cardiologist may be needed to implant such electrode. The positioning of electrodes having no direct contact to the internal organ/heart as provided herein is particularly made possible by treating the internal organ/heart via at least a DC component of an electric current.

According to one aspect of the invention, the relative orientation of the anode and the cathode is implemented such that at least a partial volume of the internal organ, such as a heart, is situated inside a geometric volume spanned between the anode and the cathode.

The volume spanned between the anode and the cathode may be the geometric volume spanned by connecting the outermost circumference of the respective electrically active parts of each of the electrodes with each other by straight lines. The volume spanned may be the volume enclosed by the electrically active areas of the respective electrically active parts and the conceived straight lines drawn between the circumferences of the electrically active areas. Any relative orientation of the electrically active parts of such cathode-anode pair spanning such volume and having at least a part of the internal organ, such as the heart, situated therein may fall into the scope of the present invention.

According to a first implementation of the present aspect, two or more implantable and/or extracorporeal electrodes may be considered. One or more of the electrodes may be used as a cathode (or alternatively as an anode), while the remaining one or more electrodes may be used as an anode (or alternatively as a counter-cathode, if the other electrode acts as anode), respectively. In that case, the volume spanned between the electrically active parts according to the invention may be understood as the sum of the volumes spanned between the cathode and the anode. In other words, the volume spanned between the anode and cathode according to the invention may be referred to the geometric volume between each cathode-anode pairing. Hence, in a configuration comprising more than two electrodes, the sum of any possible cathode-anode combination may be considered to define the volume spanned between the anode and cathode as defined according to the invention.

In a second implementation of the present aspect, only two electrodes are considered. For example, there may be a first electrode being an anode and a second electrode being a cathode (or vice versa). Then, the volume spanned between the electrically active parts according to the invention may simply be the geometric volume spanned between the electrically active part of the first electrode and the electrically active part of the second electrode.

In a third implementation of the present aspect, three electrodes may be considered. For example, there may be a first electrode being an anode, and a second and third electrode being a cathode (or vice versa). At least one of the three electrodes is placed with finite distance to the internal organ, such as a heart, while the remaining electrodes may be individually placed with either zero distance or finite distance to the internal organ, such as a heart. The volume spanned between the electrically active parts may be the sum of the geometric volumes spanned between the electrically active part of the first electrode and the electrically active part of the second electrode plus that of the electrically active part of the first electrode and the electrically active part of the third electrode.

In a fourth implementation of the present aspect, a configuration comprising four electrodes may be considered, i.e. a first, second, third and fourth electrode. In one embodiment, the first and second electrode may be cathodes, while the third and fourth electrodes may be anodes (or vice versa). At least one of the four electrodes is placed with finite distance to the internal organ, such as a heart, while the remaining electrodes may be individually placed with either zero distance or finite distance to the internal organ, such as a heart. The volume spanned between the anode and cathode may be given by the sum of volumes spanned between the first and third, the first and fourth as well as the second and third, and the second and fourth electrode.

The present aspect of the invention aims at providing a precise geometric definition of the relative orientation of the cathode and anode such that a DC electric current may more reliably flow through the internal organ, such as a heart, and thus increase the therapeutic effect.

According to a further aspect of the invention, the control unit may be implantable and positioned with finite distance to the internal organ, such as a heart. More specifically, it may be positioned subcutaneously and/or intermuscularly.

Additionally or alternatively, the control unit may be implantable and positioned submuscularly, substernally, intraabdominally, retroperitoneally and/or mediastinally.

Additionally or alternatively, at least one of the multiple electrodes may be implantable and positioned with finite distance to the internal organ, such as a heart. More specifically, it may be positioned subcutaneously and/or intermuscularly.

Additionally or alternatively, at least one of the multiple electrodes may be implantable and positioned submuscularly, substernally, intraabdominally, retroperitoneally and/or mediastinally.

Correspondingly, each of the control unit and at least one of the multiple electrodes that are being positioned with a finite distance to the internal organ, such as a heart, are independently positioned subcutaneously and/or intermuscularly.

A subcutaneous position may be understood as beneath the skin, but above the muscles. An intermuscular position may be understood as being in between muscle bundle or in between different parts of different muscle groups. In one embodiment, this is not understood as intramuscular. An electrode may be positioned both subcutaneously and intermuscularly. Generally, any subcutaneous and/or intermuscular placement of the control unit and the at least one of the multiple electrodes may be comprised as long as there is a finite distance to the internal organ, such as a heart.

Generally, the second aspect of the invention may be understood such that independent placements of each of the control unit and each of the at least one of the multiple electrodes are comprised.

In one implementation of the present aspect, the control unit may be placed subcutaneously, while a first electrode is placed intermuscularly and vice versa. Further electrodes, for example a second, third, fourth electrode etc., might be also individually placed subcutaneously and/or intermuscularly.

Correspondingly, any combination of mutual positionings as specified according to the second aspect regarding the control unit and the one or more further electrodes are comprised herein.

The present aspect of the invention aims at providing an even less invasive procedure for implanting electrodes for treatment of the internal organ, such as a heart. Subcutaneous and/or intermuscular positioning of electrodes may be advantageous in this regard as it may be comparatively easy to access these regions.

In another aspect of the invention, the control unit may be implantable and positioned with a finite distance to an internal organ, such as a heart, in a thorax region, preferably in a thorax region above the ribs/outside the rib cage, more preferably in a dorsolateral region of the left or right thorax above the ribs/outside the rib cage, in a substernal region, in a subxiphoidal region, in an abdominal region, in a lumbar region or in a sub-clavicular region, preferably in a sub-clavicular pocket, more preferably in a sub-clavicular pocket of on the left or right side of the thorax.

Additionally or alternatively, at least one of the multiple electrodes may be implantable and positioned with a finite distance to the internal organ, such as a heart, in a thorax region, preferably in a thorax region above the ribs/outside the rib cage, more preferably in a dorsolateral region of the left or right thorax above the ribs/outside the rib cage, in a substernal region, in a subxiphoidal region, in an abdominal region, in a lumbar region or in a sub-clavicular region, preferably in a sub-clavicular pocket, more preferably in a sub-clavicular pocket of on the left or right side of the thorax.

Correspondingly, each of the control unit and at least one of the multiple electrodes that are being positioned with a finite distance to an internal organ, such as a heart, are independently positioned in a thorax region, preferably in a thorax region above the ribs/outside the rib cage, more preferably in a dorsolateral region of the left or right thorax above the ribs/outside the rib cage, in a substernal region, in a subxiphoidal region, in an abdominal region, in a lumbar region or in a sub-clavicular region, preferably in a sub-clavicular pocket, more preferably in a sub-clavicular pocket of on the left or right side of the thorax

The positioning in the above specified regions may also be subcutaneous and/or intermuscular according to the foregoing aspect of the invention.

Generally, the present aspect may be understood such that independent placements of each of the control unit and each of the at least one of the multiple electrodes are comprised.

In one exemplary implementation of the present aspect, the control unit may be placed in a sub-clavicular region, in particular a sub-clavicular pocket in the left or right thorax, while a first electrode may be placed in either one of the specified positions according to the present aspect. Further electrodes, like a second, third, fourth electrode etc, may be independently placed in the same or any other of the specified positions.

Correspondingly, any combination of mutual positionings as specified according to this embodiment regarding the control unit and the one or more further electrodes are within the scope of the present aspect of the invention.

The present aspect of the invention aims at providing various possible positions for the placement of electrodes with finite distance to the internal organ, such as a heart. Different combinations of these positions may be advantageous for treating the internal organ, such as a heart, via at least a DC component of an electric current from various angles.

In a further aspect of the invention, the one or more multiple electrodes that is not positioned with finite distance to the internal organ is independently positioned inside the internal organ or positioned directly on the outside of and in direct contact with the internal organ.

In a preferred embodiment of the present aspect, the internal organ may be the heart and each of the one or more multiple electrodes that is not positioned with finite distance to the heart is independently positioned inside the heart, preferably in the right ventricle, in the coronary sinus, or in the coronary sinus and a left lateral vein of the left ventricular myocardium, or positioned directly onto the outside of the heart, preferably epicardially or pericardially on the left ventricle or on the right ventricle.

In a first implementation of the present aspect, there may only be two electrodes and the first electrode may be positioned with zero distance to the internal organ, such as a heart, while the second electrode must be positioned with finite distance to the internal organ, such as a heart, in accordance with any of the foregoing aspects of the invention.

In a second implementation of the present aspect, there may be more than two electrodes. Then, there may also be more than one electrode positioned with zero distance to the internal organ, such as a heart, as long as at least one of the other electrodes is positioned with finite distance to the internal organ/heart in accordance with any of the present and foregoing aspects of the invention.

In a third implementation of the present aspect, the electrode being positioned with zero distance to the internal organ/heart may be specifically placed directly on the outside of the internal organ/heart. In the preferred case of the internal organ being a heart, the placement on the outside may for example be epicardially or pericardially on the left ventricle or on the right ventricle. Herein, the outside of the heart may comprise any outer wall of the heart suitable for applying an electrode directly thereto. The electrode may for example be sutured to the outside of the heart.

In a fourth implementation of the present aspect, the electrode being positioned with zero distance to the internal organ/heart may also be placed inside the internal organ/heart. For example, in the case of the internal organ being a heart, the electrode may be placed in the right ventricle, in the coronary sinus, or in the coronary sinus and a left lateral vein of the left ventricular myocardium. In the latter case, an uninsulated portion of the electrode, i.e. referring to the electrically active part thereof, may be placed in the left lateral vein, while an insulated portion of the electrode, i.e. referring to the electrically inactive part thereof, may be placed in the coronary sinus region. In the case of the electrode being placed in the right ventricle, an uninsulated active part of the electrode may be located within the ventricle, while an insulated portion of the electrode may be located elsewhere. In another example, the electrode may be placed in the left ventricle.

The present aspect of the invention aims at providing an electrode assembly that combines having at least one electrode with no direct contact to the internal organ/heart with at least one electrode having direct contact with the internal organ/heart and/or being inside the internal organ/heart. This may be advantageous as it combines the reduced invasiveness and flexibility of an electrode being implanted away from the internal organ/heart with the possibility to target local sick spots of the internal organ/heart by an electrode being applied directly on or inside the internal organ/heart.

According to another aspect of the invention, one or more of the multiple electrodes is a patch electrode, preferably wherein the patch electrode is a flat patch electrode.

In a first implementation of the present aspect, the flat patch electrode may be a left ventricular flat electrode in accordance with the application document WO 2024/213655 A1, which is included by reference herein.

In a second implementation of the present aspect, only two electrodes are considered and the first electrode may be the patch electrode, while the second electrode may be a different type of electrode.

In a third implementation of the present aspect, only two electrodes are considered and both of the electrodes may be patch electrodes.

In a fourth implementation of the present aspect, three electrodes are considered and only the first electrode may be a patch electrode, while the second and third electrode may be different types of electrodes. Alternatively, the first and second electrode may be patch electrodes, while the third electrode is a different type of electrode. In a further Alternative, all three electrodes may be patch electrodes.

The present aspect of the invention aims at providing patch electrodes to be used for the implantable electrode assembly. This may be advantageous as the surface area of patch electrodes, in particular the electrically active part thereof, can be variable and thus provide greater flexibility regarding the current density that can be generated via the electrode assembly.

According to another aspect of the present invention, the internal organ may be the heart and the electrically active part of the patch electrode is positioned with finite distance to the heart such that it is directed towards the left ventricle, the right ventricle, the left atrium and/or the right atrium, or wherein the patch electrode is positioned directly onto the outside of the heart, preferably epicardially or pericardially on the left ventricle or on the right ventricle.

The electrically active part of the patch electrode being directed towards different parts of the heart may be understood such that at least a finite perpendicular vector component of an associated electrically active area of the patch electrode points towards the heart, more particularly towards one or multiple of the above specified parts of the heart.

The patch electrode may specifically be designed to be applied directly onto the outside of the heart, for example epicardially or pericardially on the left ventricle or on the right ventricle. The application onto the outside of the heart may be implemented by suturing. Additionally or alternatively, the patch electrode may also be specifically designed to be placed in any of the positions with finite distance to the heart as specified in the foregoing aspects. In these positions, the patch electrode may more specifically be applied subcutaneously and/or intermuscularly.

In a first implementation of the present aspect, only two electrodes are considered and the first electrode may be a patch electrode with finite distance to the heart such that it is directed towards the left ventricle, the right ventricle, the left atrium and/or the right atrium. This first electrode may also be extracorporeally applied to the outside of the skin. The second electrode may also be a patch electrode or alternatively a different type of electrode like a coil or finger electrode. The second electrode may further either be placed with finite distance to the heart in accordance with any of the present and foregoing aspects, or with zero distance to the heart, i.e. inside the heart, for example in the right ventricle, in the coronary sinus, or in the coronary sinus and a left lateral vein of the left ventricular myocardium, or directly on the outside of the heart, for example epicardially or pericardially on the left ventricle or on the right ventricle. This second electrode may also be extracorporeally applied to the outside of the skin.

In a second implementation of the present aspect, only two electrodes are considered and the first electrode may be a patch electrode with zero distance to the heart placed directly on the outside of the heart, for example epicardially or pericardially on the left ventricle or on the right ventricle. The second electrode may also be a patch electrode or alternatively a different type of electrode, for example a coil or finger electrode. In this implementation, the second electrode is to be placed with finite distance to the heart such that it is directed towards the left ventricle, the right ventricle, the left atrium and/or the right atrium. This second electrode may also be extracorporeally applied to the outside of the skin.

In a third implementation of the present aspect, three electrodes are considered. The first electrode may be a patch electrode placed with finite distance to the heart such that it is directed towards the left ventricle, the right ventricle, the left atrium and/or the right atrium. This first electrode may also be extracorporeally applied to the outside of the skin. The second and third electrode may either be patch electrodes or different type of electrodes. The second and third electrodes may be placed with zero distance to the heart, i.e. inside the heart or directly on the outside of the heart, for example epicardially or pericardially on the left ventricle or on the right ventricle. Alternatively in this configuration, only one of the second and third electrodes may be placed with zero distance to the heart, while the other electrode may be placed with finite distance to the heart. In another alternative, both of the second and third electrodes may be placed with finite distance to the heart. Each of the second and third electrode may be either a patch electrode or a different type of electrode, like a coil or finger electrode. Further, one or both of the second and third electrodes may also be extracorporeally applied to the outside of the skin.

In a fourth implementation of the present aspect, three electrodes are considered. The first electrode may be a patch electrode placed with zero distance to the heart, i.e. directly on the outside of the heart, for example epicardially or pericardially on the left ventricle or on the right ventricle. Either one of the second and third electrodes is placed with finite distance to the heart such that it is directed towards the left ventricle, the right ventricle, the left atrium and/or the right atrium. Alternatively in this configuration, both of the second and third electrodes may be placed with finite distance to the heart. Further, one or both of the second and third electrode may also be extracorporeally applied to the outside of the skin.

The present aspect of the invention aims at providing an electrode assembly that utilizes patch electrodes being implanted either away from the heart or applied directly on the heart or a combination of these two options. This may be advantageous as patch electrodes can be used for both purposes and thus provide for various current distributions possible for treatment of the heart via at least a DC component of the electric current.

According to yet another aspect of the invention, one or more of the multiple electrodes may be a finger electrode or a coil electrode, preferably wherein the finger electrode or coil electrode may be a single finger/coil electrode or a multiple finger/coil electrode.

The coil electrode may be understood as an electrode having wound wires. A finger electrode may in the broadest sense be understood as a longitudinal piece of electrically conducting material, for example a piece of wire of a certain thickness. The cross section of piece of wire may be flat or round and/or be comprised of strand. A single finger or coil electrode may comprise only a single segment of an electrically active part of such electrode, for example an uninsulated portion exposing electrically conducting material. A multiple coil electrode may comprise multiple coil segments, each of which form an electrically active part of the electrode that are separated by electrically inactive portions therebetween. For example, the coil segments may be uninsulated segments exposing wound electrical wires with insulated portions therebetween. A multiple finger electrode may be configured as an electrode having multiple branches of longitudinal pieces of electrically conducting wire, wherein the individual branches may be referred to segments branching off of a common wire.

In a first implementation of the present aspect, only two electrodes are considered and the first electrode may be the finger or coil electrode, while the second electrode may be a different type of electrode, for example a patch electrode.

In a second implementation of the present aspect, only two electrodes are considered and both of the electrodes may be either finger or coil electrodes, or any combination thereof.

In a third implementation of the present aspect, three electrodes are considered and only the first electrode may be a finger or coil electrode, while the second and third electrode may be different types of electrodes, for example patch electrodes. Alternatively, the first and second electrode may be finger or electrodes, while the third electrode is a different type of electrode, for example a patch electrode. In a further Alternative, all three electrodes may be either finger or coil electrodes, or any combination thereof.

The present aspect of the invention aims at providing an electrode assembly that utilizes finger or coil electrodes to be used in the implantable electrode assembly. This may be advantageous as finger or coil electrodes are specifically suitable for transvenous implanting inside the heart. Implanting electrodes inside the heart can be particularly beneficial for treating certain areas of the heart specifically.

According to a further aspect of the invention, the internal organ is the heart and the electrically active part of the finger/coil electrode is positioned inside the heart, more preferably in the right ventricle, in the coronary sinus or in the coronary sinus and a left lateral vein of the left ventricular myocardium, or wherein the finger/coil electrode is positioned with a finite distance to the heart, preferably wherein the electrically active part of the finger/coil electrode is directed towards left ventricle, the right ventricle, the left atrium and/or the right atrium.

The electrically active part of the finger/coil electrode being directed towards different parts of the heart may be understood such that at least a finite perpendicular vector component of an associated electrically active area of the patch electrode points towards the heart, more particularly towards one or multiple of the above specified parts of the heart. For example, in case the coil or finger electrode has a cylindrical shape, multiple perpendicular vectors may be present around the whole circumference of the cylinder shape while always being perpendicular to the longitudinal axis of the cylinder.

The finger or coil electrode may specifically be designed to be applied inside the heart, more particularly in the right ventricle, in the coronary sinus or in the coronary sinus and a left lateral vein of the left ventricular myocardium. The implantation of the finger or coil electrode inside the heart may be implemented transvenously.

A coil/finger electrode being placed inside the heart may experience an induced movement due to the beating of the heart. In case one of the electrodes in the electrode assembly is a coil or finger electrode situated inside the heart, the induced current path or current distribution that is generated between the coil/finger electrode inside the heart and a second electrode in a different position with finite distance to the heart may be effectively enhanced by this induced movement. Correspondingly, using an electrode assembly according to the present invention that features placing one coil/finger electrode inside the heart may effectively enhance the effective current distribution suitable to treat a heart. Similarly, the same effect may also enhance the geometric volume spanned between such coil/finger electrode inside the heart and one or more additional electrodes.

Additionally or alternatively, the finger or coil electrode may also be specifically designed to be placed in any of the positions with finite distance to the heart as specified in the foregoing aspects. In these positions, the finger or coil electrode may more specifically be applied subcutaneously and/or intermuscularly.

In a first implementation of the present aspect, only two electrodes are considered and the first electrode may be a finger or coil electrode with zero distance to the heart, in particular inside the heart, more particularly in the right ventricle, in the coronary sinus or in the coronary sinus and a left lateral vein of the left ventricular myocardium. The second electrode may also be a finger or coil electrode or alternatively be a different type of electrode, for example a patch electrode. The second electrode is to be placed with finite distance to the heart such that it is directed towards the left ventricle, the right ventricle, the left atrium and/or the right atrium. This second electrode may also be also placed subcutaneously and/or intermuscularly. Alternatively, this second electrode may also be extracorporeally applied to the outside of the skin, where it may preferably be a patch electrode.

In a second implementation of the present aspect, only two electrodes are considered and the first electrode may be a finger or coil electrode with finite distance to the heart such that it is directed towards the left ventricle, the right ventricle, the left atrium and/or the right atrium. The second electrode may also be a finger or coil electrode or alternatively a different type of electrode, for example a patch electrode. In this implementation, the second electrode may be placed with either finite distance to the heart, such that it is directed towards the left ventricle, the right ventricle, the left atrium and/or the right atrium, or with zero distance to the heart, in particular inside the heart, more particularly in the right ventricle, in the coronary sinus or in the coronary sinus and a left lateral vein of the left ventricular myocardium. In the case that the second electrode is a patch electrode, it may also be extracorporeally applied to the outside of the skin. Alternatively, this second electrode may also be also placed subcutaneously and/or intermuscularly.

In a third implementation of the present aspect, three electrodes are considered. The first electrode may be a finger or coil electrode placed with zero distance to the heart, in particular inside the heart, more particularly in the right ventricle, in the coronary sinus or in the coronary sinus and a left lateral vein of the left ventricular myocardium. The second and third electrode may either be finger or coil electrodes or different type of electrodes, for example a patch electrode. Either one of the second and third electrodes is to be placed with finite distance to the heart such that it is directed towards the left ventricle, the right ventricle, the left atrium and/or the right atrium. Alternatively in this configuration, both of the second and third electrodes may be placed with finite distance to the heart. Further, one or both of the second and third electrodes may be patch electrodes and may be extracorporeally applied to the outside of the skin. Alternatively, this second and third electrode may also be also placed subcutaneously and/or intermuscularly.

In a fourth implementation of the present aspect, three electrodes are considered. The first electrode may be a finger or coil electrode placed with finite distance to the heart such that it is directed towards the left ventricle, the right ventricle, the left atrium and/or the right atrium. The second and third electrode may either be finger or coil electrodes or different types of electrodes, for example a patch electrode. The second and third electrodes may be placed with zero distance to the heart, in particular inside the heart, more particularly in the right ventricle, in the coronary sinus or in the coronary sinus and a left lateral vein of the left ventricular myocardium, or directly onto the outside of the heart, for example epicardially or pericardially on the left ventricle or on the right ventricle. Alternatively in this configuration, only one of the second and third electrodes may be placed with zero distance to the heart, while the other electrode may be placed with finite distance to the heart. In another alternative, both of the second and third electrodes may be placed with finite distance to the heart. Further, the first electrode may be extracorporeally applied to the outside of the skin. Alternatively, this first electrode may also be also placed subcutaneously and/or intermuscularly.

The present aspect of the invention aims at providing an electrode assembly that utilizes coil or finger electrodes to be implanted either away from the heart or applied directly inside the heart. This may be advantageous as finger or coil electrodes can be used for both purposes and thus provide for various current distributions possible for treatment of the heart via at least a DC component of the electric current.

According to another aspect of the invention, the internal organ is the heart and the partial volume of the heart may be at least a partial volume of the left ventricle, a partial volume of the left heart muscle, a partial volume of the right ventricle, a partial volume of the left atrium and/or a partial volume of the right atrium during atrial and/or ventricular diastole.

Considering the partial volume of the heart during atrial and/or ventricular diastole ensures that the heart volume is well defined. Correspondingly, the partial volume of the heart as used in the present invention may be considered as a partial volume with respect to the maximum possible heart volume.

According to a further aspect of the invention, the partial volume of the left ventricle, the left heart muscle, the right ventricle, the left atrium and/or the right atrium may be at least 10%, 20%, 30%, 40%, 50% 60%, 70%, 80%, 90% or 100% of the total volume of the left ventricle, the left heart muscle, the right ventricle, the left atrium and/or the right atrium during atrial and/or ventricular diastole.

The present and foregoing aspect of the invention aims at providing an electrode assembly that specifies certain areas of the heart to be specifically intended for treatment by at least the DC component of the electric current. This may be advantageous as certain areas of the heart may be particularly affected by certain heart diseases.

According to a further aspect of the invention, the electric current is a direct current or a quasi-stationary electric current without AC components.

A direct current or quasi-stationary electric current without AC-components may be referred to as a pure direct current.

According to a yet another aspect of the invention the DC component of the electric current is maintained below a threshold value of normal physiological excitation of muscular tissue being permeated by the induced current path.

The threshold value of normal physiological excitation of muscular tissue may be referred to as a voltage value above which the muscular tissue is excited and thus contracts. According to the present aspect of the invention, the DC component of the potential difference/voltage inducing the DC component of the electric current between cathode and anode may be below said threshold.

Generally, the threshold voltage value for normal physiological excitation of muscular tissue may depend strongly on the electrode geometry and/or their relative orientation with respect to the muscular tissue's position. However, it is well within the common knowledge of a person skilled in the art to deduce threshold voltage values for certain electrode assemblies and positions. It is thus within the standard capability of the skilled person to operate the electrode assembly in accordance with the current aspect of the invention.

The threshold value of normal physiological excitation of muscular tissue may be additionally or alternatively referred to as a current or current density value above which the muscular tissue is excited and thus contracts. According to the present aspect of the invention, the DC component of the electric current induced by the DC component of the applied voltage between cathode and anode may be below said threshold.

Similarly to the case when considering voltage thresholds, the threshold current value or current density value for normal physiological excitation of muscular tissue may also depend on the electrode geometry and/or their relative orientation with respect to the internal organs's position. However, it is also well within the common knowledge of a person skilled in the art to deduce threshold current or current density values for certain electrode assemblies and positions. It is thus within the standard capability of the skilled person to operate the electrode assembly in accordance with the current aspect of the invention.

Generally, this threshold may also be dependent on the type of electrodes used, the distance between the electrodes and the particular placement of the respective electrodes, in particular the relative orientation of the cathode and anode around the internal organ. However, when specifically the current density is used as a threshold value, it might be less dependent on said conditions.

In one implementation of the present aspect, the DC component is being kept below said current or voltage threshold and may be further modulated with an additional AC component. The sum of the DC component and the peak amplitude of the AC component of voltage or current may either be below or above the threshold value for normal physiological excitation of a cardiac muscle.

According to another aspect of the invention, the internal organ is the heart and the DC component of the electric current is maintained below a threshold value of normal physiological excitation of the cardiac muscle.

The threshold value of normal physiological excitation of the cardiac muscle may be referred to as a voltage value above which the cardiac muscle is excited and thus contracts. According to the present aspect of the invention, the DC component of the potential difference/voltage inducing the DC component of the electric current between cathode and anode may be below said threshold.

Generally, the threshold voltage value for normal physiological excitation of the cardiac muscle may depend strongly on the electrode geometry and/or their relative orientation with respect to the heart's position. However, it is well within the common knowledge of a person skilled in the art to deduce threshold voltage values for certain electrode assemblies and positions. It is thus within the standard capability of the skilled person to operate the implantable and/or extracorporeal electrode assembly in accordance with the current aspect of the invention.

The threshold value of normal physiological excitation of the cardiac muscle may be additionally or alternatively referred to as a current or current density value above which the cardiac muscle is excited and thus contracts. According to the present aspect of the invention, the DC component of the electric current induced by the DC component of the applied voltage between cathode and anode may be below said threshold.

Similarly to the case when considering voltage thresholds, the threshold current value or current density value for normal physiological excitation of the cardiac muscle may also depend on the electrode geometry and/or their relative orientation with respect to the heart's position. However, it is also well within the common knowledge of a person skilled in the art to deduce threshold current or current density values for certain electrode assemblies and positions. It is thus within the standard capability of the skilled person to operate the implantable and/or extracorporeal electrode assembly in accordance with the current aspect of the invention.

Generally, this threshold may also be dependent on the type of electrodes used, the distance between the electrodes and the particular placement of the respective electrodes, in particular the relative orientation of the cathode and anode around the heart. However, when specifically the current density is used as a threshold value, it might be less dependent on said conditions.

In one implementation of the present aspect, the DC component is being kept below said current or voltage threshold and may be further modulated with an additional AC component. The sum of the DC component and the peak amplitude of the AC component of voltage or current may either be below or above the threshold value for normal physiological excitation of a cardiac muscle.

According to yet another aspect of the invention, the electric current is a DC or a quasi-stationary electric current without AC components.

The current amperage or current density of a pure DC electric current may have no or only negligible changes in time. A quasi-stationary electric current may be in accordance with the definition of DC as used herein. It particularly may not have any AC components as defined herein.

The present and foregoing aspect of the invention aims at providing an electrode assembly using a DC component of an electric current or a pure DC current that is below a threshold value of normal physiological excitation of the cardiac muscle. This may be advantageous since the treatment of the heart according to the present invention is particularly effective when using direct current with sub-threshold current density values.

According to a further aspect of the invention, the power supply unit is either part of the control unit or separate to the control unit, wherein in case of a separate power supply unit, the power supply unit is either implanted or external.

In a first implementation according to the present aspect, the power supply unit may be a battery.

In a second implementation according to the present aspect, the power supply unit may be external and thus be connected by a power connector line to the implanted control unit.

The present aspect of the invention aims at providing a control unit that is configurable to have its power supply either integrated therein or external. An integrated power supply unit may be advantageous since it does not require a patient to carry the external power supply. An external power supply, on the other hand, may be beneficial since it can be much bigger in size, does not have to be biocompatible and may have more charging capacity.

According to another aspect of the invention, the control unit comprises a printed circuit board assembly, PCBA, preferably a flexible PCBA.

According to a further aspect of the invention, one of the multiple electrodes and the control unit are configured as an integrated entity.

An integrated entity may be understood as any kind of structural integration of the control unit with one of the multiple electrodes. Structural integration may comprise loose connections, detachable connections, modular connections, flexible connections and rigid connections of the two parts. Further, structural integration may comprise glueing, fastening via connection elements like screws, nails and the like, embedding in polymers or any other kind of fixing two elements to each other. Structural integration of an electrode and a control unit may also comprise to use sections of the control unit itself as an electrode. The type of electrode used for structural integration may not be restricted. Hence, the control unit may be integrated for example with a finger, coil or patch electrode.

The integrated entity may be positioned with a finite distance to the internal organ, such as a heart, in accordance with the respective foregoing aspects of the invention. Such integrated entity may be implantable and thus implanted within the human body.

The present aspect of the invention aims at providing a control unit that is used in conjunction with an electrode. This may be advantageous as it effectively reduces the number of components and thus may also reduce the number of components to be implanted in a patient and is thus even less invasive.

According to yet another aspect of the invention, the integrated entity is implemented by structurally mounting one of the multiple electrodes to a surface of a housing of the control unit, preferably wherein the electrode is an electrically conductive mesh, more preferably a mesh comprising metal, or a metal plate.

In embodiments where a metallic mesh used, the mesh density may be between 1000 meshes/cm² and 5000 meshes/cm², preferably between 2000 meshes/cm² and 4000 meshes/cm², more preferably between 3000 meshes/cm² and 4000 meshes/cm². In particular embodiments, the mesh size or mesh density may be about 3600 meshes/cm² or alternatively 3.481 meshes/cm².

In another embodiment, the metallic mesh used may be defined using the unit “Mesh” which is commonly known to a skilled person. In this embodiment, the metallic mesh may be of between 50 and 500 Mesh, preferably between 100 and 200 Mesh, more preferably about 150 Mesh.

In a first implementation of the present aspect, a patch electrode may be structurally mounted to the surface of the housing of the control unit. Alternatively, a finger or coil electrode may be mounted thereto. Structural mounting may comprise anything that is not merely loosely connected to each other. In particular, it may comprise rigid detachable connections, glueing, fastening via connection elements like screws, nails and the like, embedding in polymers or any other kind of fixing two elements to each other.

In a second implementation of the present aspect, the electrode being mounted to the surface of the housing of the control unit may be an electrically conductive mesh, in particular a mesh comprising metal, more particularly a metal mesh.

In an alternative embodiment of the second implementation, the electrode being mounted to the surface of the housing of the control unit may be a metal plate. The present aspect of the invention aims at providing a way of integrating one of the electrodes with the control unit in a structural way. This may be advantageous as the integration may be more durable and safe.

According to a further aspect of the invention, the integrated entity may be implemented by configuring the electrically active part of one of the multiple electrodes as an integrated subsection of the housing of the control unit, wherein the integrated subsection may be at least partially constituted of the housing of the control unit, preferably wherein the subsection may be constituted of the complete housing of the control unit.

The integrated subsection of the housing of the control unit in accordance with the present aspect of the invention may be understood as a part of the housing and is thus intended for protecting the electronic components within the housing.

In a first implementation of the present aspect, the integrated subsection of the housing may be an electrically conductive part that is electrically insulated with respect to the rest of the housing of the control unit. More specifically, the integrated subsection may be an electrically conductive part comprising an electrically conductive mesh, preferably a mesh comprising metal, even more preferably a metal mesh, or a metal plate. The metal of the mesh or the metal plate may be made of a material with high corrosive resistance, preferably it may comprise PtIr or be completely made of PtIr.

In a second implementation of the present aspect, the housing of the control unit may comprise titanium or may be made mainly or exclusively of titanium.

In a third implementation of the present aspect, when the integrated subsection may be constituted of the complete housing of the control unit, the integrated subsection/housing may be mainly made of titanium and may be configured as (part of) the cathode of the implantable and/or extracorporeal electrode assembly.

The present aspect of the invention aims at providing a control unit that simultaneously works as an electrode. This may be advantageous as it omits the necessity to combine two components, i.e. the control unit and an electrode, with each other but rather uses an existing component (the control unit) to fulfill the function of acting as an electrode.

According to yet another aspect of the invention, the integrated entity is implemented by configuring one of the multiple electrodes as an envelope or pouch into which the control unit is inserted.

In a first implementation of the present aspect, the envelope or pouch electrode may be configured as a patch electrode. In particular, the patch electrode may comprise an opening suitable to incorporate the control unit therein.

In a second implementation of the present aspect, the envelope or pouch electrode may be configured as an electrically conductive mesh having an opening suitable to incorporate the control unit therein. The mesh may preferably be a mesh comprising metal, even more preferably a metal mesh. The mesh may be made of an electrically conducting material with high corrosive resistance, preferably it may comprise PtIr or be completely made of PtIr.

In a preferred embodiment, only one side of the envelope or pouch may be comprised of the metal mesh or metal plate. The opposite side of the control unit may be an electrically non-conducting material like for example silicone. The electrically non-conducting side of the envelope or pouch may further preferably be pointing in an outward direction of the body. In this way, there may be no conducting material like a metallic mesh covering the control unit on this particular side. Otherwise, when using a control unit having a radio transceiver, the capability of wirelessly communicating with an external device for remote programming and readout of data may be suppressed.

The present aspect of the invention aims at providing a control unit combined with an electrode that still uses two separate components. This may be advantageous as it omits the necessity to combine two components structurally or integrating an electrode functionality to the control unit, but rather provides a modular and detachable way of forming an integrated entity from a control unit and an electrode.

According to a further aspect of the invention, the integrated entity is implemented by embedding the control unit together with one of the multiple electrodes in a hard polymer and/or a flexible polymer.

The present aspect of the invention aims at providing another way of combining a control unit with an electrode to form an integrated entity. This may be advantageous since embedding in a polymer provides a method that is capable of forming an integrated entity that is mostly independent of the size and shape of either the control unit or the electrode.

According to the invention, a Portable User Terminal (PUT) configured for programming of the control unit and readout of data recorded by the control unit may be provided, wherein the control unit is in accordance with the implantable electrode assembly according to any of the aforementioned aspects of the invention.

### BRIEF DESCRIPTION OF THE DRAWINGS

The present disclosure will be more readily appreciated by reference to the following detailed description when being considered in connection with the accompanying drawings.

FIG. 1 is an illustration schematically showing the placement of an electrode assembly according to a preferred embodiment of the invention in which a control unit 30 is placed subcutaneously and/or intermuscularly in a sub-clavicular region of the left thorax, a patch electrode 20 is placed subcutaneously and/or intermuscularly in a dorsolateral region of the left thorax directed towards the left ventricle and a coil electrode 10 is placed inside the right ventricle of the heart.

FIG. 2 is an illustration schematically showing the placement of an electrode assembly according to a preferred embodiment of the invention in which a control unit 30 is placed subcutaneously and/or intermuscularly in a sub-clavicular region of the left thorax, a patch electrode 20 is placed directly on the outside of the left ventricle and a further patch electrode 10 is placed subcutaneously and/or intermuscularly in a dorsolateral region in the right thorax next to the right ventricle and directed towards the right ventricle.

FIG. 3 is an illustration schematically showing the placement of an electrode assembly according to a preferred embodiment of the invention in which a control unit 30 is placed subcutaneously and/or intermuscularly in a sub-clavicular region of the left thorax, a patch electrode 20 is placed subcutaneously and/or intermuscularly in a dorsolateral region in the left thorax directed towards the left ventricle and a further patch electrode 10 is placed subcutaneously and/or intermuscularly in a region in the right thorax next to the right ventricle and directed thereto.

FIG. 4 is an illustration schematically showing the placement of an electrode assembly according to a preferred embodiment of the invention in which a control unit 30 is placed subcutaneously and/or intermuscularly in a sub-clavicular region of the left thorax, a coil electrode 20 is placed inside the coronary sinus and a left lateral vein of the left ventricular myocardium and a further patch electrode 10 is placed subcutaneously and/or intermuscularly in a dorsolateral region in the right thorax next to the right ventricle and directed thereto.

FIG. 5 is an illustration schematically showing the placement of an electrode assembly according to a preferred embodiment of the invention in which a control unit 30 is placed subcutaneously and/or intermuscularly in a sub-clavicular region of the left thorax, a coil electrode 20 is placed inside the coronary sinus and a left lateral vein of the left ventricular myocardium and a further patch electrode 10 is placed in a subxiphoidal area below the apex of the heart.

FIG. 6 is an illustration schematically showing the placement of an electrode assembly according to a preferred embodiment of the invention in which a control unit 30 is placed subcutaneously and/or intermuscularly in a sub-clavicular region of the right thorax, a patch electrode 20 is placed subcutaneously and/or intermuscularly in a sub-clavicular region of the left thorax and a further patch electrode 10 is placed directly on the outside of the right ventricle of the heart.

FIG. 7 is an illustration schematically showing the placement of an electrode assembly according to a preferred embodiment of the invention in which a control unit 30 is placed subcutaneously and/or intermuscularly in a sub-clavicular region of the left thorax, a patch electrode 20 is placed subcutaneously and/or intermuscularly in a dorsolateral region in the left thorax directed towards the left ventricle, a further patch electrode 10 is placed subcutaneously and/or intermuscularly in a dorsolateral region of the right thorax next to the right ventricle and directed thereto and a further coil electrode 70 is placed inside the right ventricle of the heart.

FIG. 8 is an illustration schematically showing the placement of an electrode assembly according to a preferred embodiment of the invention in which a control unit 30 and a patch electrode 20 form an integrated entity 60 that is placed subcutaneously and/or intermuscularly in a sub-clavicular region of the left thorax and a coil electrode 10 is placed inside the right ventricle of the heart.

FIG. 9 is an illustration schematically showing the placement of an electrode assembly according to a preferred embodiment of the invention in which the housing of a control unit 30 is being configured as an electrode 20 thus forming an integrated entity 60 that is placed subcutaneously and/or intermuscularly in a sub-clavicular region of the left thorax and a coil electrode 10 is placed inside the right ventricle of the heart.

FIG. 10 is an illustration schematically showing the placement of an electrode assembly according to a preferred embodiment of the invention in which the housing of a control unit 30 is being configured as an electrode 20 and thus forming an integrated entity 60 is placed subcutaneously and/or intermuscularly in a sub-clavicular region of the left thorax and a coil electrode 10 is placed in a subxiphoidal area below the heart.

FIG. 11A is an illustration schematically showing the geometric volume spanned between the electrically active part 22 of a patch electrode and the electrically active part 12 of a finger/coil electrode.

FIG. 11B is an illustration schematically showing the geometric volume spanned between the electrically active part 22 of a patch electrode and the electrically active part 12 of another patch electrode.

FIG. 12 is an illustration schematically showing the placement of an electrode assembly according to a preferred embodiment of the invention with an extracorporeal control unit 100, wherein an extracorporeal patch electrode 80 is placed on the outside of the skin in a dorsolateral region of the left thorax directed towards the left ventricle and a coil electrode 10 is placed inside the right ventricle of the heart.

FIG. 13 is an illustration schematically showing the placement of an electrode assembly according to a preferred embodiment of the invention with an extracorporeal control unit 100, wherein an extracorporeal patch electrode 80 is placed on the outside of the skin in a dorsolateral region of the left thorax directed towards the left ventricle and a coil electrode 20 is placed inside the left ventricle of the heart.

FIG. 14 is an illustration schematically showing the placement of an electrode assembly according to a preferred embodiment of the invention with an extracorporeal control unit 100, wherein a first extracorporeal patch electrode 80 is placed on the outside of the skin in a dorsolateral region of the left thorax directed towards the left ventricle, a second extracorporeal patch electrode 90 is placed on the outside of the skin in a dorsolateral region of the right thorax directed towards the right ventricle and a coil electrode 10 is placed inside the right ventricle of the heart.

FIG. 15 is an illustration schematically showing the placement of an electrode assembly according to a preferred embodiment of the invention with an extracorporeal control unit 100, wherein a first extracorporeal patch electrode 80 is placed on the outside of the skin in a dorsolateral region of the left thorax directed towards the left ventricle, a second extracorporeal patch electrode 90 is placed on the outside of the skin in a dorsolateral region of the right thorax directed towards the right ventricle and a coil electrode 20 is placed inside the left ventricle of the heart.

### DETAILED DESCRIPTION OF PREFERRED EMBODIMENTS

In the following, the invention will be explained in more detail with reference to the accompanying figures. In the figures, like elements are denoted by identical reference numerals.

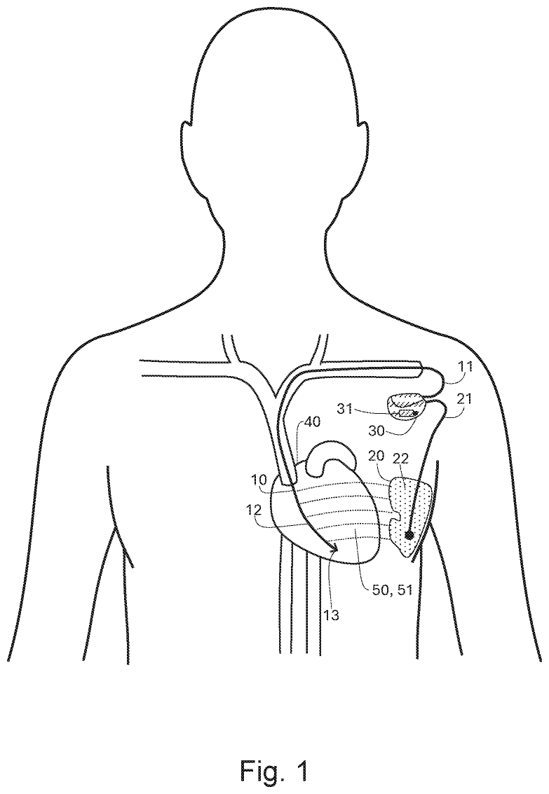

FIG. 1 shows a preferred embodiment of the present invention in which an electrode assembly with a control unit 30, a first electrode 10 and a second electrode 20 is shown.

The control unit 30 comprises an integrated power supply unit 31 and is placed subcutaneously and/or intermuscularly in a sub-clavicular region of the left thorax. It may alternatively also be placed subcutaneously and/or intermuscularly in a sub-clavicular region of the right thorax or in another suitable position within the thorax, abdominal or lumbar regions. In the particular embodiment shown here, the control unit 30 may be placed in a so-called pocket that is present in the sub-clavicular region and is suitable to take up the control unit 30 therein.

The first electrode 10 is a finger or coil electrode that is connected to the control unit 30 by a connector line 11. The finger/coil electrode 10 comprises an electrically active part 12 that is placed transvenously inside the right ventricle of a heart 40. Further, the coil electrode 10 comprises an attachment means 13 that is configured as an anchor in order to fix the electrode to the heart, in this case to the inside of the right ventricle. The electrically active part 12 may be an uninsulated portion in order to allow electric current to flow from or to the coil electrode 10.

The second electrode 20 is a patch electrode with an electrically active part 22 that is connected to the control 30 by a connector line 21. The electrically active part 22 of the patch electrode 20 may be the entire or only a portion of the total area of the patch electrode 20. Further, the electrically active area 22 may be situated only on one side of the patch electrode 20. However, a patch electrode according to the present invention may also comprise electrically active areas 22 on both sides of the patch. The electrically active area 22 of the patch electrode 20 may be an uninsulated portion that allows electric current to flow from or to the patch electrode 20. The patch electrode in the shown configuration is placed subcutaneously and/or intermuscularly in a dorsolateral region in the left thorax directed towards left ventricle. The area of the electrically active part 22 is oriented such that at least a finite component of a perpendicular (geometric) vector of the area is directed towards at least a part of the heart 40. More particularly, the area may be oriented such that the perpendicular vector component is directed towards at least one of the left ventricle, the right ventricle, the left atrium and/or the right atrium.

In the shown configuration, a potential difference/voltage is applied between the first electrode 10 and the second electrode 20 such that an electric current is induced therebetween. Either of the electrodes may be an anode or a cathode depending on the sign of the applied voltage. In the shown embodiment, the electric current dominantly passes through the left ventricle.

The patch electrode 20 and coil electrode 10 according to the shown embodiment illustrate an exemplary relative orientation in which the induced current path 50 between cathode and anode passes through at least a partial volume of the heart 40. In the shown configuration, the current path 50 is particularly intended to pass through the left ventricle of the heart 40, thereby medically treating the heart 40.

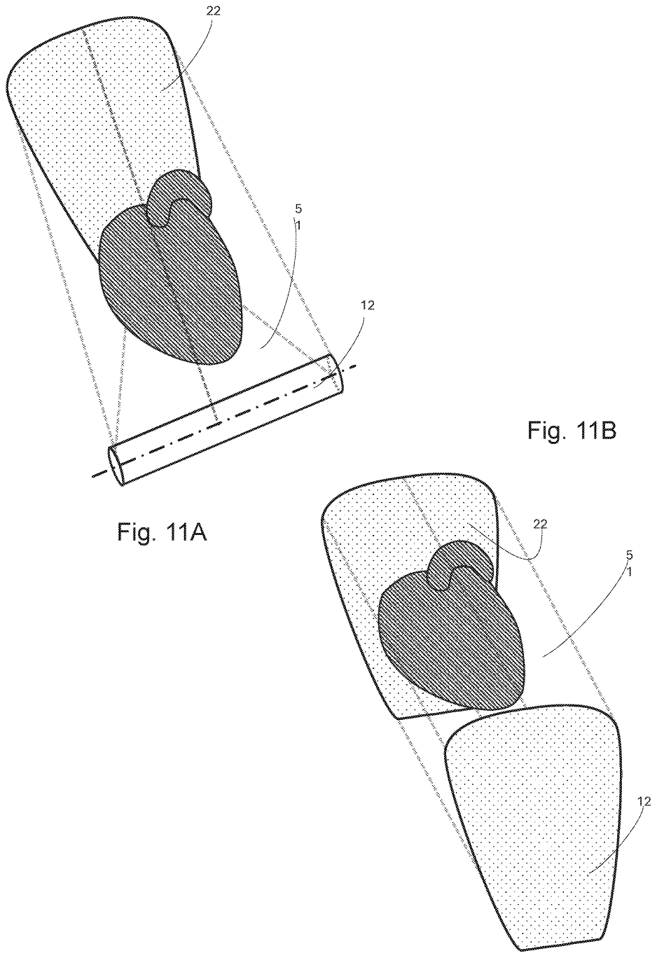

The electrodes 10, 20 further define a geometric volume 51 spanned between the electrically active part 12 of the first electrode 10 and the electrically active part 22 of the second electrode 20. An exemplary illustration of such volume 51 spanned therebetween may be gathered from FIG. 11A. Therein, the geometric volume 51 is defined by drawing straight lines between the edges of the electrically active area 22 of the patch electrode to the edges of the electrically active part 12 of the coil electrode. The electrically active part 12 of the coil electrode is exemplarily shown as a cylinder 12. Accordingly, the volume 51 may be defined by drawing straight lines from the outermost points of the electrically active part 22 of the patch electrode to the outermost points of the cylinder 12. Generally, the outermost points of each of the electrically active parts which are connected to each other by straight lines may be defined as those points that are still just visible from the perspective of the respective opposite electrode.

There may be multiple alternative electrode configurations based on the example of FIG. 1. For example, the patch electrode 20 may also be a coil or finger electrode. Alternatively, the patch electrode 20 of FIG. 1 may also be placed in a left abdominal region and oriented such that the electrically active part 22 of the electrode 20 is directed towards the heart 40, in particular towards the right ventricle, left ventricle, right atrium and/or left atrium. In another alternative of FIG. 1, the coil electrode 10 may be placed substernally. In particular, it may be placed in a longitudinal direction along the sternum.

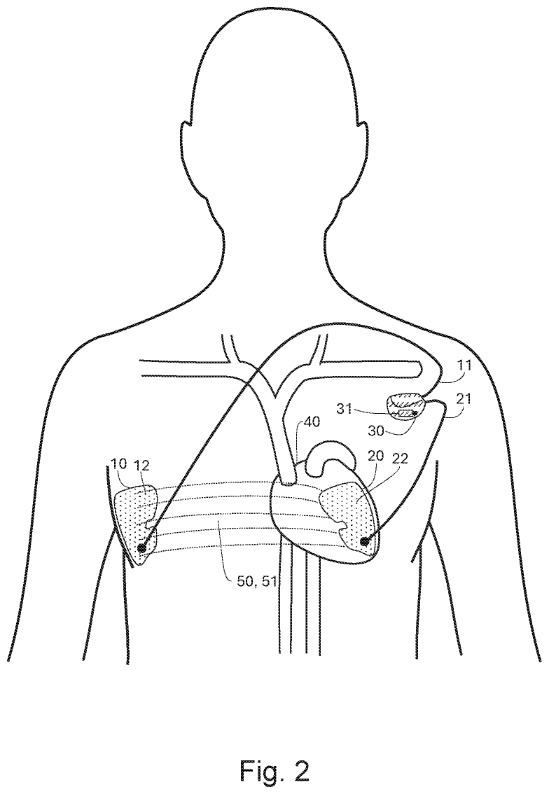

FIG. 2 shows a further preferred embodiment of the invention in which an electrode assembly with a control unit 30, a first electrode 10 and a second electrode 20 is shown.

The control unit 30 is placed identically compared to the embodiment shown in FIG. 1.

The first electrode 10 is a patch electrode that is connected to the control unit 30 by a connector line 11. The patch electrode 10 according to this embodiment is placed subcutaneously and/or intermuscularly in a dorsolateral region of the right thorax at a height that corresponds to the height of the right ventricle.

The second electrode 20 is another patch electrode that is connected to the control 30 by a connector line 21. The patch electrode 20 is placed with zero distance to the heart 40, in particular on the outside of the left ventricle. The patch electrode 20 may be applied epicardially or pericardially.

In the shown embodiment, the electric current passes dominantly through the right and left ventricle, thereby medically treating the heart 40. A slight shift of the electrodes 10 and 20 in either up- or downward direction may be used to influence the current path 50 to treat different parts of the heart 40 such as the left ventricle, the right ventricle, the left atrium and/or the right atrium.

The electrodes 10, 20 of FIG. 2 further define a geometric volume 51 spanned between the electrically active part 12 of the first electrode 10 and the electrically active part 22 of the second electrode 20. An exemplary illustration of such volume 51 spanned therebetween may be gathered from FIG. 11B. Therein, the geometric volume 51 is defined by drawing straight lines between the edges of the electrically active area 22 of the patch electrode to the edges of the electrically active part 12 of another patch electrode. Accordingly, the volume 51 may be defined by drawing straight lines from the outermost points of the electrically active part 22 of the patch electrode to the outermost points of the electrically active part 12 of the other patch electrode 10. Generally, the outermost points of each of the electrically active parts which are connected to each other by straight lines may be defined as those points that are still just visible from the perspective of the respective opposite electrode.

There may be multiple alternative electrode configurations based on the example of FIG. 2. For example, the patch electrode 10 may also be a coil or finger electrode. Alternatively, the patch electrode 10 of FIG. 2 may also be placed in a sub-clavicular region of the right thorax and may be oriented such that the electrically active part 12 of the electrode 10 is directed towards the heart, in particular towards the right ventricle, left ventricle, right atrium and/or left atrium. Alternatively, the patch electrode 10 of FIG. 2 may also be placed in a right abdominal region and may be oriented such that the electrically active part 12 of the electrode 10 is directed towards the heart, in particular towards the right ventricle, left ventricle, right atrium and/or left atrium.

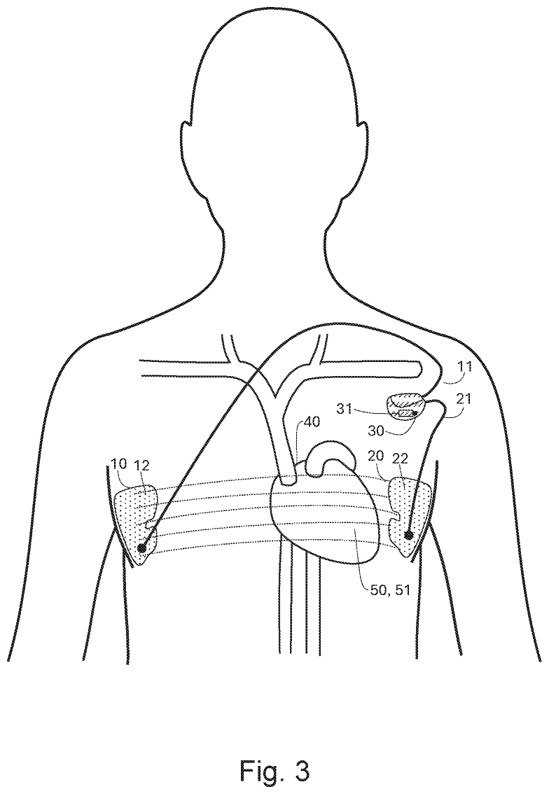

FIG. 3 shows a further preferred embodiment of the invention in which an electrode assembly with a control unit 30, a first electrode 10 and a second electrode 20 is shown.

The embodiment of FIG. 3 shows an identical placement of the control unit 30 and of the patch electrode 10 as compared to the embodiment shown in FIG. 2. Different to the embodiment of FIG. 2, the second electrode 20 is a patch electrode that is placed subcutaneously and/or intermuscularly in a dorsolateral region of the right thorax in a height corresponding to the height of the left ventricle.

In the shown embodiment, the electric current path 50 passes dominantly through the right and left ventricle, but may also be oriented such that it passes through other combinations of different parts of the heart 40. This may be realized by a slight shift of the electrodes 10 and 20 in either up- or downward direction to influence the current path 50 to treat different parts of the heart 40 such as the left ventricle, the right ventricle, the left atrium and/or the right atrium.

The electrodes 10, 20 of FIG. 3 define a geometric volume 51 in a similar manner as shown in FIG. 11B as described above.

There may be multiple alternative electrode configurations based on the example of FIG. 3. For example, either one or both of the patch electrodes 10 and 20 may also be coil or finger electrodes. Alternatively, the patch electrode 10 of FIG. 3 may also be placed in a sub-clavicular region of the right thorax and may be oriented such that the electrically active part 12 of the electrode 10 is directed towards the heart 40, in particular towards the right ventricle, left ventricle, right atrium and/or left atrium. In doing so, the patch electrode 20 may simultaneously be placed in a left abdominal region and may be oriented such that the electrically active part 12 of the electrode 10 is directed towards the heart 40, in particular towards the right ventricle, left ventricle, right atrium and/or left atrium. The described replacement may also be implemented the other way round. In a further alternative, the first electrode 10 may be coil or finger electrode that is placed substernally, in particular in a longitudinal direction along the sternum.

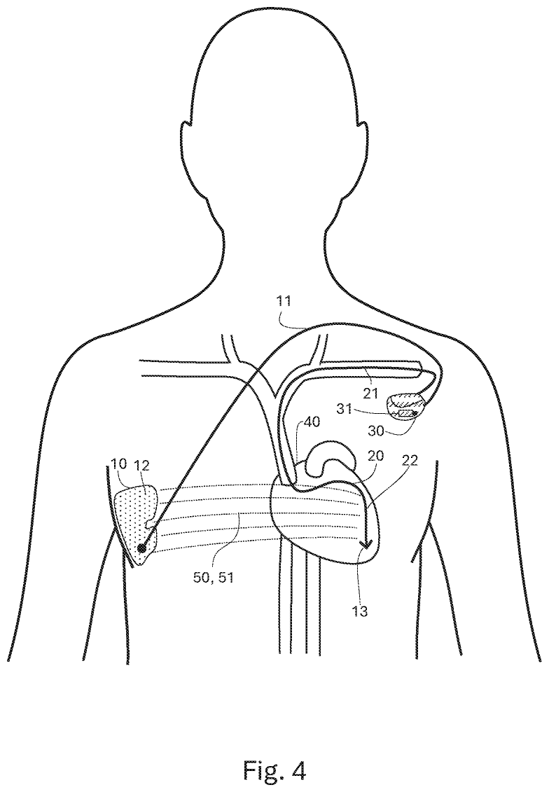

FIG. 4 shows a further preferred embodiment of the invention in which an electrode assembly with a control unit 30, a first electrode 10 and a second electrode 20 is shown.

The embodiment of FIG. 4 shows an identical placement of the control unit 30 and of the patch electrode 10 as compared to the embodiment shown in FIG. 3 or FIG. 2. Different to the embodiment of FIG. 2 or 3, the second electrode 20 is a coil or finger electrode that is placed in the coronary sinus and a left lateral vein of the left ventricular myocardium. Preferably, the coil or finger electrode comprises an electrically active part 22 that is placed in the left lateral vein, wherein the part of the electrode situated in the coronary sinus may be electrically inactive, for example an insulated part.

In the shown embodiment, the electric current path 50 passes dominantly through the right and left ventricle. Different parts of the heart or at least different current distributions across the heart may be realized by shifting the patch electrode 10 either up- or downward direction to influence the current path 50. Alternatively or additionally, the patch electrode 10 may be shifted posterior or anterior. When shifting the electrode within the body, the electrically active part 12 may be reoriented such that it is always directed towards the heart or different parts of the heart. In this way, arbitrary current paths/distributions 50 may be realized through the heart 40.

The electrodes 10, 20 of FIG. 4 define a geometric volume 51 in a similar manner as shown in FIG. 11A as described above.

There may be multiple alternative electrode configurations based on the example of FIG. 4. For example, the patch electrode 10 may also be coil or finger electrode.

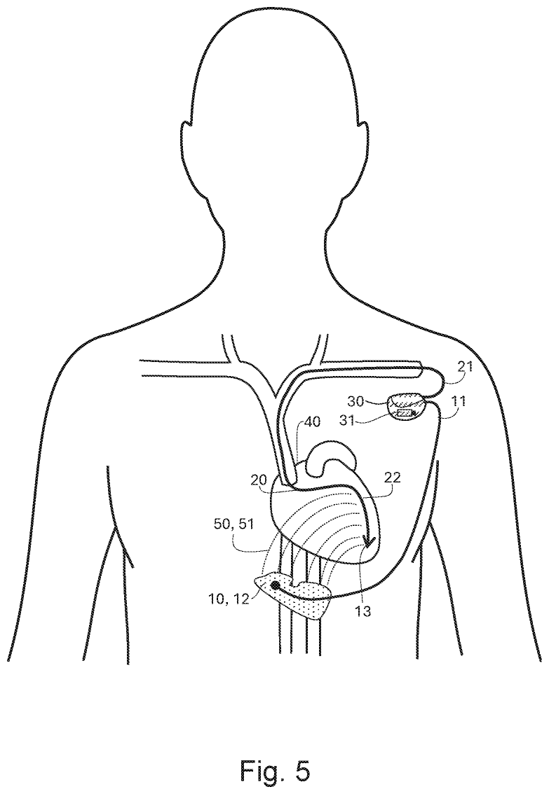

FIG. 5 shows a further preferred embodiment of the invention in which an electrode assembly with a control unit 30, a first electrode 10 and a second electrode 20 is shown.

The embodiment of FIG. 5 shows an identical placement of the control unit 30 and of the coil electrode 20 as compared to the embodiment shown in FIG. 4. Different to the embodiment of FIG. 4, the first electrode 10 is a patch electrode that is placed in a sub-xiphoidal region below the apex of the heart 40. The patch electrode may also be placed subcutaneously and/or intermuscularly.

In the shown embodiment, the electric current path 50 passes dominantly through the right and left ventricle. Different parts of the heart or at least different current distributions across the heart 40 may be realized by shifting the patch electrode 10 in a lateral left or right direction in the transversal plane to influence the current path 50. Alternatively or additionally, the patch electrode 10 may be shifted posterior or anterior in the transversal plane. When shifting the patch electrode 10 within the body, the electrically active part 12 may be reoriented such that it is always directed towards the heart 40 or different parts of the heart 40. In this way, arbitrary current paths/distributions 50 may be realized through the heart 40.

The electrodes 10, 20 of FIG. 5 define a geometric volume 51 in a similar manner as shown in FIG. 11A as described above.

There may be multiple alternative electrode configurations based on the example of FIG. 5. For example, the patch electrode 10 may also be coil or finger electrode. In this case, the coil electrode 10 may be placed below the apex of the heart 40 or in front of the heart 40. In a further alternative, the first electrode 10 may be coil or finger electrode that is placed substernally, in particular in a longitudinal direction beneath the sternum.

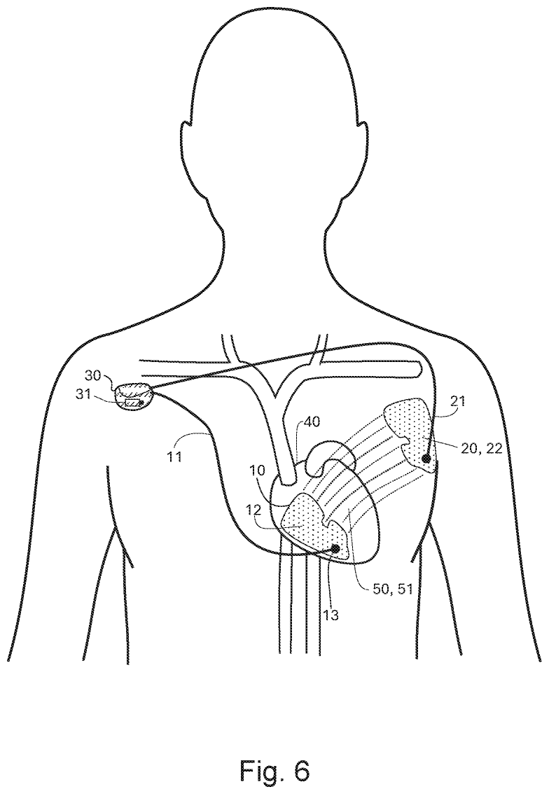

FIG. 6 shows a further preferred embodiment of the invention in which an electrode assembly with a control unit 30, a first electrode 10 and a second electrode 20 is shown.

The control unit 30 is placed subcutaneously and/or intermuscularly in a sub-clavicular region of the right thorax. It may alternatively also be placed subcutaneously and/or intermuscularly in a sub-clavicular region of the left thorax or in another suitable position within the thorax, abdominal or lumbar regions. In the particular embodiment shown here, the control unit 30 may be placed in a so-called pocket that is present in the sub-clavicular region and is highly suitable to take up the control unit 30 therein.

The first electrode 10 is a patch electrode that is connected to the control unit 30 by a connector line 11. The patch electrode 10 is placed directly on the outside of the right ventricle and may be applied epicardially or pericardially.

The second electrode 20 is another patch electrode that is placed subcutaneously and/or intermuscularly in a sub-clavicular region of the left thorax. The area of the electrically active part 22 is oriented such that at least a finite component of a perpendicular vector of the area is directed towards the heart 40. More particularly, the area may be oriented such that the perpendicular vector component is directed towards at least one of the left ventricle, the right ventricle, the left atrium and/or the right atrium.

In the shown embodiment, the electric current path 50 passes dominantly through the left atrium and the right ventricle. Different parts of the heart or at least different current distributions across the heart may be realized by shifting the patch electrode 20 in a lateral left or right direction in the transversal plane to influence the current path 50. Alternatively or additionally, the patch electrode 10 may be shifted posterior or anterior in the transversal plane. When shifting the patch electrode 10 within the body, the electrically active part 12 may be reoriented such that it is always directed towards the heart or different parts of the heart. In this way, arbitrary current paths/distributions 50 may be realized through the heart 40.

The electrodes 10, 20 of FIG. 6 define a geometric volume 51 in a similar manner as shown in FIG. 11B as described above.

There may be multiple alternative electrode configurations based on the example of FIG. 6. For example, the patch electrode 20 may also be coil or finger electrode.

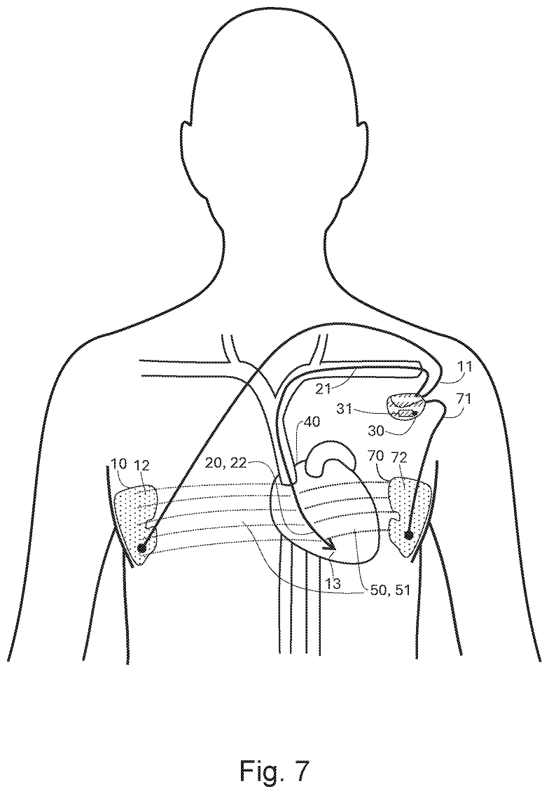

FIG. 7 shows a further preferred embodiment of the invention in which an electrode assembly with a control unit 30, a first electrode 10, a second electrode 20 and a third electrode 70 is shown.

The embodiment of FIG. 7 shows an identical placement of the control unit 30 and of the patch electrode 10 as compared to FIGS. 2-4. Different to the embodiment of FIG. 2-4, the second electrode 20 is a coil or finger electrode that is placed in the right ventricle. Additionally, the shown assembly comprises a third electrode 70 being a patch electrode that is placed subcutaneously and/or intermuscularly in a dorsolateral region of the left thorax. The third electrode has an electrically active part 72 that is directed towards the heart 40.

In the shown embodiment, the electric current path 50 is strongly dependent on which of the electrodes is used as a cathode or anode. In this example, we assume the first patch electrode 10 and the third patch electrode 70 to be the cathode, while the second coil electrode 20 is the anode. Assuming this configuration, the (technical) DC component of the electric current flow may be both from the second coil electrode 20 towards the first patch electrode 10 and from the second coil electrode 20 towards the third patch electrode 70.

In the shown embodiment, the electric current dominantly passes through the right and left ventricle. Different parts of the heart or at least different current distributions across the heart may be realized by shifting the patch electrodes 10 and 70 in either up- or downward direction to influence the current path 50. Alternatively or additionally, the patch electrodes 10 and 70 may be shifted posterior or anterior. When shifting the electrodes 10 and 70 within the body, the electrically active part 12 and 72 may be reoriented such that they are always directed towards the heart or towards different parts of the heart 40. In this way, arbitrary current paths/distributions 50 may be realized through the heart 40.

The electrodes 10 and 20 forming one cathode-anode pair define a geometric volume 51 in a similar manner as shown in FIG. 11A as described above. Similarly, the electrodes 20 and 70 forming another cathode-anode pair define a geometric volume 51 in a similar manner as shown in FIG. 11A as described above. The geometric volume spanned between the cathode and the anode may be the sum of the geometric volumes of both cathode-anode pairs.

There may be multiple alternative electrode configurations based on the example of FIG. 7. For example, either one or both of the patch electrode 10 and 70 may also be coil or finger electrodes. Either one or both of the first electrode 10 and third electrode 70 may also be placed subcutaneously and/or intermuscularly in an abdominal region, a lumbar region, a sub-clavicular region or a sub-xiphoidal region. Additionally or alternatively, the coil electrode 20 may also be placed in the coronary sinus or a coronary sinus and a left lateral vein of the left ventricular myocardium. In a further alternative based on FIG. 7, the second electrode 20 may be a coil or finger electrode that is placed substernally. In particular, it may be placed longitudinally along the sternum.

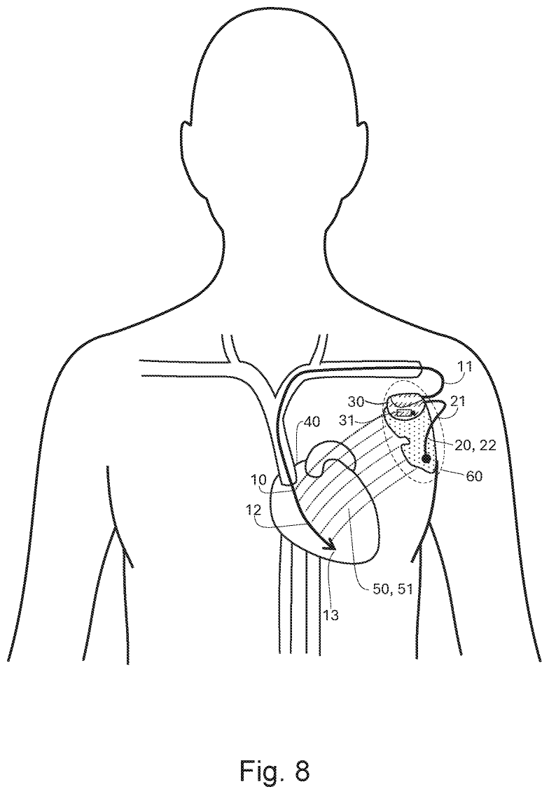

FIG. 8 shows a further preferred embodiment of the invention in which an electrode assembly with a control unit 30, a first electrode 10 and a second electrode 20 is shown.

The embodiment of FIG. 8 shows an identical placement of the control unit 30 and of the coil electrode 10 as compared to FIG. 1. Different to the embodiment of FIG. 1, the second electrode 20 is a patch electrode forming an integrated entity 60 with the control unit 30. The integrated entity 60 may be configured according to any of the specified aspects and implementations regarding the integrated entity 60 as described herein.

In the shown embodiment, the patch electrode 20 may be structurally mounted to the surface of the housing of the control unit 30. Alternatively, the second electrode 20 may be a finger or coil electrode that is mounted to the control unit 30.

The electrode 20 being mounted to the surface of the housing of the control unit 30 may alternatively be an electrically conductive mesh, in particular a mesh comprising metal, more particularly a metal mesh, or alternatively a metal plate.

In another embodiment of the integrated entity 60 as shown in FIG. 8, the patch electrode 20 may be configured as an envelope or pouch. In particular, the patch electrode 20 may comprise an opening suitable to incorporate the control unit 30 therein.

In another embodiment of the integrated entity 60 as shown in FIG. 8, the patch electrode 20 may be configured as an envelope or pouch into which the control unit 20 may be inserted and the envelope or pouch electrode may be configured as an electrically conductive mesh having an opening suitable to incorporate the control unit 30 therein.

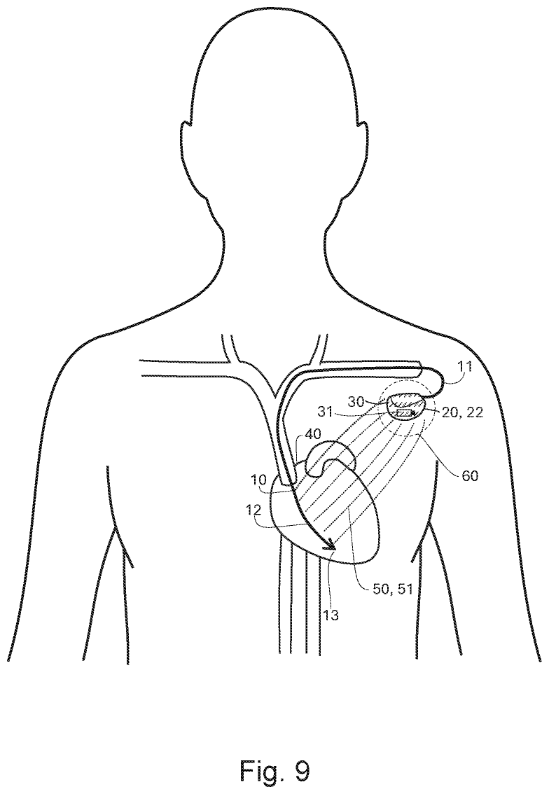

FIG. 9 shows a further preferred embodiment of the invention in which an electrode assembly with a control unit 30, a first electrode 10 and a second electrode 20 is shown.

The embodiment of FIG. 9 shows an identical placement of the control unit 30 and of the coil electrode 10 as compared to FIG. 8. Different to the embodiment of FIG. 8, the second electrode 20 is formed by at least an integrated subsection of the control unit 30 itself, thus forming an integrated entity 60.

In the shown embodiment, the integrated subsection is constituted of the entire housing of the control unit 30. The integrated subsection/housing may be mainly made of titanium and may be configured as the cathode of the implantable electrode assembly.

In another embodiment according to FIG. 9, the integrated subsection of the housing may be an electrically conductive part that is electrically insulated with respect to the rest of the housing of the control unit 30. More specifically, the integrated subsection may be an electrically conductive part comprising an electrically conductive mesh, preferably a mesh comprising metal, even more preferably a metal mesh, or alternatively a metal plate. The metal of the mesh or metal plate may be of a material with high corrosive resistance, preferably it may comprise PtIr or be completely made of PtIr.

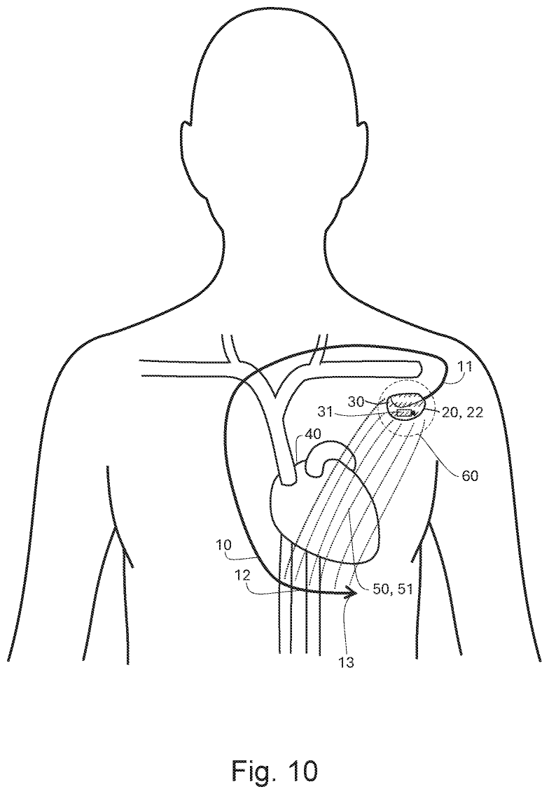

FIG. 10 shows a further preferred embodiment of the invention in which an electrode assembly with a control unit 30, a first electrode 10 and a second electrode 20 is shown.

The embodiment of FIG. 10 shows an identical placement of the integrated entity 60 forming an electrode 20 as compared to FIG. 9. Different to the embodiment of FIG. 9, the first electrode 10 is a coil or finger electrode placed in a subxiphoidal area below of the heart. Alternatively, the electrically active part 12 coil electrode 10 may be placed directly beneath the apex of the heart 40 or in front of the heart 40.

In the shown embodiment, the electric current dominantly passes through the right and left ventricle. Different parts of the heart 40 or at least different current distributions across the heart 40 may be realized by shifting the coil electrodes 10 in either up- or downward direction to influence the current path 50. Alternatively or additionally, the coil or finger electrode 10 may be shifted left or right in the transversal plane and/or posterior or anterior in the transversal plane. When shifting the coil/finger electrode 10 within the body, the electrically active part 12 may be reoriented such that they are always directed towards the heart 40 or towards different parts of the heart 40. In this way, arbitrary current paths/distributions 50 may be realized through the heart 40.

There may be multiple alternative electrode configurations based on the example of FIG. 10. For example, the coil electrode 10 may also be a patch electrode. In the latter case, the patch electrode 10 may be placed below the apex of the heart.

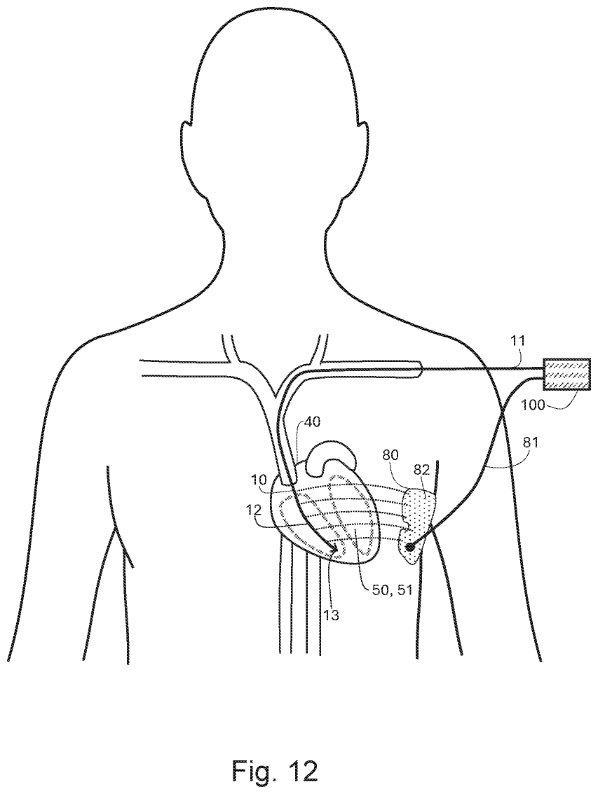

FIG. 12 shows a preferred embodiment of the invention in which an electrode assembly with an extracorporeal control unit 100, a first electrode 10 and a second electrode 80 is shown.

The control unit 100 is arranged extracorporeally. It may comprise an integrated power supply unit or may be powered by external means (not shown).

The first electrode 10 is a finger or coil electrode that is connected to the control unit 100 by a connector line 11. The finger/coil electrode 10 comprises an electrically active part 12 that is placed transvenously inside the right ventricle of a heart 40. Further, the coil electrode 10 comprises an attachment means 13 that is configured as an anchor in order to fix the electrode to the heart, in this case to the inside of the right ventricle. The electrically active part 12 may be an uninsulated portion in order to allow electric current to flow from or to the coil electrode 10. Such electrode may be combined with any suitable right ventricular heart-assist devices known to the skilled person.

Alternatively, the first electrode 10 may also be placed in the left ventricle. The placement may be implemented transarterially.

The second electrode 80 is an extracorporeal patch electrode with an electrically active part 82 that is connected to the control unit 100 by a connector line 81. The extracorporeal patch electrode 80 in the shown configuration is attached to the outside of the skin in a dorsolateral region of the left thorax and directed towards left ventricle. The area of the electrically active part 82 is oriented such that at least a finite component of a perpendicular (geometric) vector of the area is directed towards at least a part of the heart 40. More particularly, the area may be oriented such that the perpendicular vector component is directed towards at least one of the left ventricle, the right ventricle, the left atrium and/or the right atrium.

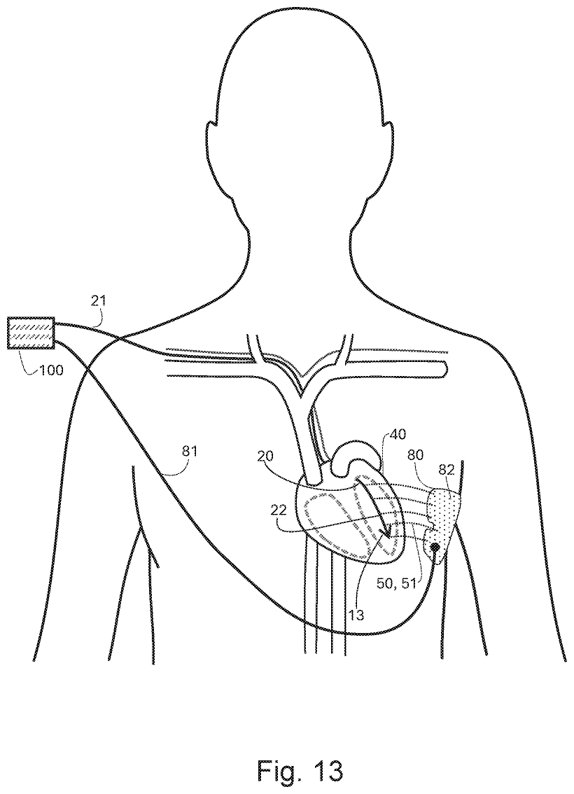

FIG. 13 shows a preferred embodiment of the invention in which an electrode assembly with an extracorporeal control unit 100, a first electrode 20 and a second electrode 80 is shown.

The control unit 100 is arranged extracorporeally. It may comprise an integrated power supply unit or may be powered by external means (not shown).

The first electrode 20 may be a coil or finger electrode placed in the left ventricle of the heart 40. The placement of the electrode may be implemented transarterially. Such electrode may be combined with any suitable left ventricular heart-assist devices known to the skilled person.

The second electrode 80 is an extracorporeal patch electrode with identical features and placement as the second electrode 80 of the embodiment of FIG. 12.

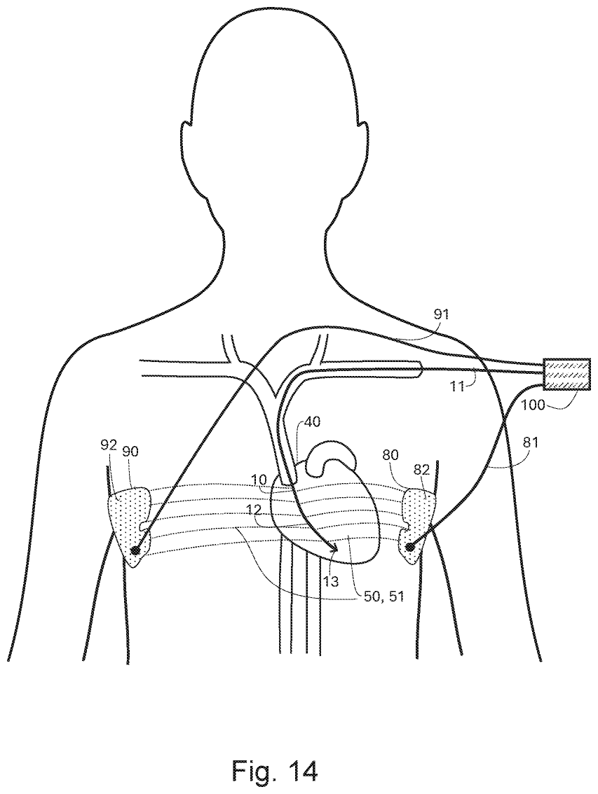

FIG. 14 shows a preferred embodiment of the invention in which an electrode assembly with an extracorporeal control unit 100, a first electrode 10, a second electrode 80 and a third electrode 90 is shown.

The control unit 100 is arranged extracorporeally. It may comprise an integrated power supply unit or may be powered by external means (not shown).

The first electrode 10 is a finger or coil electrode that is connected to the control unit 100 by a connector line 11. The finger/coil electrode 10 comprises an electrically active part 12 that is placed transvenously inside the right ventricle of a heart 40. Further, the coil electrode 10 comprises an attachment means 13 that is configured as an anchor in order to fix the electrode to the heart, in this case to the inside of the right ventricle. The electrically active part 12 may be an uninsulated portion in order to allow electric current to flow from or to the coil electrode 10. Such electrode may be combined with any suitable right ventricular heart-assist devices known to the skilled person.

The second electrode 80 is an extracorporeal patch electrode with an electrically active part 82 that is connected to the control unit 100 by a connector line 81. The extracorporeal patch electrode 80 in the shown configuration is attached to the outside of the skin in a dorsolateral region of the left thorax and directed towards left ventricle. The area of the electrically active part 82 is oriented such that at least a finite component of a perpendicular (geometric) vector of the area is directed towards at least a part of the heart 40. More particularly, the area may be oriented such that the perpendicular vector component is directed towards at least one of the left ventricle, the right ventricle, the left atrium and/or the right atrium.

The third electrode 90 is an extracorporeal patch electrode with an electrically active part 92 that is connected to the control unit 100 by a connector line 91. The extracorporeal patch electrode 90 in the shown configuration is attached to the outside of the skin in a dorsolateral region of the right thorax and directed towards right ventricle. The area of the electrically active part 92 is oriented such that at least a finite component of a perpendicular (geometric) vector of the area is directed towards at least a part of the heart 40. More particularly, the area may be oriented such that the perpendicular vector component is directed towards at least one of the left ventricle, the right ventricle, the left atrium and/or the right atrium.

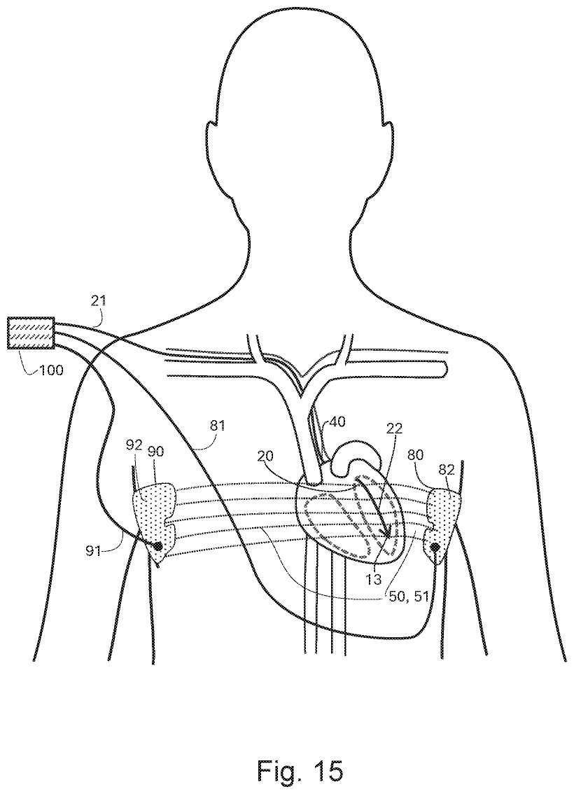

FIG. 15 shows a preferred embodiment of the invention in which an electrode assembly with an extracorporeal control unit 100, a first electrode 20, a second electrode 80 and a third electrode 90 is shown.

The control unit 100 is arranged extracorporeally. It may comprise an integrated power supply unit or may be powered by external means (not shown).

The first electrode 20 may be a coil or finger electrode placed in the left ventricle of the heart 40. The placement of the electrode may be implemented transarterially. Such electrode may be combined with any suitable left ventricular heart-assist devices known to the skilled person.

The second electrode 80 and the third electrode 90 are extracorporeal patch electrodes with identical features and placement as the second and third electrodes 80, 90 of the embodiment of FIG. 14.

All embodiments of the present invention as described herein are considered to be combinable in any combination, unless the person skilled in the art considers such a combination to be technically impractical.

### LIST OF REFERENCE NUMERALS

First electrode 10
Connector line of the first electrode 11
Electrically active part of the first electrode 12
Attachment means 13
Second electrode 20
Connector line of the second electrode 21
Electrically active part of the second 22 electrode
Control unit 30
Power Supply unit 31
Heart 40
Induced current path between anode and 50 cathode
Volume spanned between anode and cathode 51
Integrated entity 60
Third electrode 70
Connector line of the third electrode 71
Electrically active part of the third electrode 72
First extracorporeal electrode 80
Connector line of first extracorporeal 81 electrode 82
Active part of first extracorporeal electrode 90
Second extracorporeal electrode 91
Connector line of second extracorporeal 92 electrode
Active part of second extracorporeal electrode

1. An implantable electrode assembly comprising:
multiple electrodes, the multiple electrodes comprising an implantable first electrode and at least one implantable additional electrode, wherein each of the electrodes comprises an electrically active part; and
a control unit being supplied with power by a power supply unit, wherein the control unit is electrically connected to each of the electrodes;
wherein the control unit is configured to establish a potential difference between an anode and a cathode;
the anode is represented by one or more of the electrically active parts of the multiple electrodes and the cathode is represented by one or more of the remaining electrically active parts of the multiple electrodes, such that an electric current with a finite direct current (DC) component is induced between the anode and the cathode, the control unit being configured to set the electric current to a preset value by regulating the potential difference between the anode and the cathode; and
the control unit and at least one of the multiple electrodes are positioned with a finite distance to a heart, wherein the relative orientation of the anode and the cathode is implemented such that at least a partial volume of the heart is situated inside an induced electric current path between the anode and the cathode.

2. The implantable electrode assembly of claim 1, wherein the relative orientation of the anode and the cathode is implemented such that at least a partial volume of the heart is situated inside a geometric volume spanned between the anode and the cathode.

3. The implantable electrode assembly of claim 1, wherein each of the control unit and at least one of the multiple electrodes that is being positioned with a finite distance to the heart is independently positioned subcutaneously and/or intermuscularly.

4. The implantable electrode assembly of claim 1, wherein each of the control unit and at least one of the multiple electrodes that is being positioned with a finite distance to the heart is independently positioned in a thorax region, in a substernal region, in subxiphoidal region, in an abdominal region, in a lumbar region or in a subclavicular region.

5. The implantable electrode assembly of claim 1, wherein each of the one or more multiple electrodes that is not positioned with finite distance to the heart is independently positioned inside the heart.

6. The implantable electrode assembly of claim 1, wherein the each of the one or more multiple electrodes that is not positioned with finite distance to the heart is independently positioned directly on the pericardium of the heart.

7. The implantable electrode assembly of claim 1, wherein one or more of the multiple electrodes is a patch electrode.

8. The implantable electrode assembly of claim 7, wherein the electrically active part of the patch electrode is positioned with finite distance to the heart such that it is directed towards the left ventricle, the right ventricle, the left atrium and/or the right atrium.

9. The implantable electrode assembly of claim 6, wherein the electrically active part of the patch electrode is positioned directly on the pericardium of the heart.

10. The implantable electrode assembly of claim 1, wherein one or more of the multiple electrodes is a finger electrode or a coil electrode.

11. The implantable electrode assembly of claim 10, wherein the electrically active part of the finger/coil electrode is positioned inside the heart.

12. The implantable electrode assembly of claim 10, wherein the electrically active part of the finger/coil electrode is positioned with a finite distance to the heart.

13. The implantable electrode assembly of claim 1, wherein the partial volume of the heart is at least a partial volume of the left ventricle, a partial volume of the left heart muscle, a partial volume of the right ventricle, a partial volume of the left atrium and/or a partial volume of the right atrium during atrial and/or ventricular diastole.

14. The implantable electrode assembly of claim 1, wherein the DC component of the electric current is maintained below a threshold value of normal physiological excitation of the cardiac muscle.

15. The implantable electrode assembly of claim 1, wherein one of the multiple electrodes and the control unit are configured as an integrated entity.

16. The implantable electrode assembly of claim 14, wherein the integrated entity is implemented by structurally mounting one of the multiple electrodes to a surface of a housing of the control unit.

17. The implantable electrode assembly of claim 14, wherein the integrated entity is implemented by configuring one of the multiple electrodes as an integrated subsection of the housing of the control unit, wherein the integrated subsection is at least partially constituted of the housing of the control unit.

18. The implantable electrode assembly of claim 14, wherein the integrated entity is implemented by configuring one of the multiple electrodes as an envelope or pouch into which the control unit is inserted.

19. The implantable electrode assembly of claim 14, wherein the integrated entity is implemented by embedding the control unit together with one of the multiple electrodes in a hard polymer and/or a flexible polymer.

20. A Portable User Terminal (PUT) configured for programming of the control unit and readout of data recorded by the control unit, wherein the control unit is in accordance with the implantable electrode assembly of claim 1.
